SEQUENCE LISTING (22 sequences)

SEQ ID NO: 1 - protein, 50 aa, Artificial Sequence
  Synthetic
  MISC_FEATURE (6)..(50): may be absent
GSGGSGSGGSGSGGSGSGGSGSGGSGSGGSGSGGSGSGGSGSGGSGSGGS

SEQ ID NO: 2 - protein, 40 aa, Artificial Sequence
  Synthetic
  MISC_FEATURE (5)..(40): may be absent
GGGSGGGSGGGSGGGSGGGSGGGSGGGSGGGSGGGSGGGS

SEQ ID NO: 3 - protein, 4 aa, Artificial Sequence
  Synthetic
GGGG

SEQ ID NO: 4 - protein, 5 aa, Artificial Sequence
  Synthetic
GGGGG

SEQ ID NO: 5 - protein, 4 aa, Artificial Sequence
  Synthetic
GGSG

SEQ ID NO: 6 - protein, 5 aa, Artificial Sequence
  Synthetic
GGSGG

SEQ ID NO: 7 - protein, 5 aa, Artificial Sequence
  Synthetic
GSGSG

SEQ ID NO: 8 - protein, 5 aa, Artificial Sequence
  Synthetic
GSGGG

SEQ ID NO: 9 - protein, 5 aa, Artificial Sequence
  Synthetic
GGGSG

SEQ ID NO: 10 - protein, 5 aa, Artificial Sequence
  Synthetic
GSSSG

SEQ ID NO: 11 - protein, 19 aa, Artificial Sequence
  Synthetic
MGWSLILLFLVAVATRVLS

SEQ ID NO: 12 - protein, 31 aa, Artificial Sequence
  Synthetic
SAWSHPQFEKGGGSGGGSGGSSAWSHPQFEK

SEQ ID NO: 13 - protein, 4 aa, Artificial Sequence
  Synthetic
GGGS

SEQ ID NO: 14 - protein, 366 aa, Artificial Sequence
  Synthetic
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLIDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 15 - protein, 6 aa, Artificial Sequence
  Synthetic
ENLYFQ

SEQ ID NO: 16 - protein, 256 aa, Artificial Sequence
  Synthetic
GGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRA

SEQ ID NO: 17 - protein, 639 aa, Artificial Sequence
  Synthetic
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSHHHHHHHHGS

SEQ ID NO: 18 - protein, 216 aa, Artificial Sequence
  Synthetic
LTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 19 - protein, 224 aa, Artificial Sequence
  Synthetic
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 239 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - protein, 14 aa, Artificial Sequence
  Synthetic
GGGSHHHHHHHHGS

SEQ ID NO: 22 - protein, 841 aa, Artificial Sequence
  Synthetic
SAWSHPQFEKGGGSGGGSGGSSAWSHPQFEKGGGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLIDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSENLYFQGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRAGGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

(19) United States
(12) Patent Application Publication
(10) Pub. No.: US 2021/0188971 A1
(43) Pub. Date: Jun. 24, 2021

(54) MONOCLONAL ANTIBODY FUSIONS

(71) Applicant: Quidel Corporation, San Diego, CA (US)

(72) Inventors: Jason J. Sun, San Diego, CA (US); Richard L. Egan, Oceanside, CA (US); Jason McClure, San Diego, CA (US); Werner Kroll, Wayland, MA (US)

(21) Appl. No.: 17/127,810

(22) Filed: Dec. 18, 2020

Related U.S. Application Data
(60) Provisional application No. 62/950,397, filed on Dec. 19, 2019.

(51) Int. Cl. C07K 16/26; C12N 9/02; C07K 16/28; G01N 33/535; C07K 14/72

(52) U.S. Cl. CPC C07K 16/26; C12Y 113/12007; C12N 9/0069; C07K 16/2869; C07K 2319/61; C07K 14/721; C07K 2319/30; C07K 2317/55; G01N 33/535

(57) ABSTRACT

Antibody fusions comprising any antibody or fragment thereof, operably linked, and expressed as a fusion with a label of interest are provided. Plasmids, vectors, host cells, methods, and kits related to the described antibody fusions are also provided. The antibody fusions are useful at least in the diagnostic and reagent setting.

### CROSS-REFERENCE TO RELATED APPLICATIONS

This application claims the benefit of U.S. Provisional Application No. 62/950,397, filed Dec. 19, 2019, which is incorporated by reference herein.

### TECHNICAL FIELD

The present disclosure generally relates to the field of antibodies and antibody diagnostics, and, in particular, antibody fusions with a reagent of interest.

### BACKGROUND

Current antibody production and testing techniques involve expression of an antibody of interest, ensuring that the antibody has proper folding, glycosylation and the like. Subsequent to antibody expression, the antibody must be purified and processed, often resulting in loss of a certain amount of antibody. For diagnostic and other uses, the antibody is often conjugated to a protein (such as a tag or label) of interest or to a solid support (such as a microbead, nitrocellulose, or plastic such as polystyrene), thereby exposing the antibody to often harsh conjugation conditions. This can have a negative impact on the antibody-label product. There is a need in the art to produce labelled antibody fusions while obviating or avoiding the above drawbacks.

The present disclosure provides for antibody fusions which overcome the shortcomings of these techniques, resulting in increased yield, ease of manufacture, and reducing production costs. The antibody is recombinantly expressed with a protein reagent of interest, and with a tag fused to the antibody. Thus, the detection reagent need not be “conjugated” post-expression, eliminating the need for post-purification processing with loss of antibody or the need to expose the MAb to harsh conjugation conditions.

### BRIEF SUMMARY

In one aspect, recombinant monoclonal antibodies (Mabs) for immunoassays each of which has a fusion protein reagent recombinantly expressed and fused to the antibody of interest are described. The antibodies can include any antibody of interest. In some embodiments, antibodies of interest may include, e.g., anti-procalcitonin (PCT) Mabs, TSHR-specific M22 Mabs, Lyme Mabs, Flu ANP/BNP and RSV ANP Mabs and others. Protein reagents which can be expressed and fused to the antibody of interest can include, e.g., include enzymes, fluorescent proteins, covalent like attachment reagents, linker reagents and the like. The fusion antibodies provide improved functionality along with reduced production cost. This is achieved by recombinantly expressing a protein reagent with a tag fused to the antibody such that no additional step or effort is required to conjugate the detection reagent, thus eliminating the need for post purification processing. This avoids the usual loss of antibody as well as avoiding the need to expose the MAb to harsh conjugation conditions.

In one embodiment, a monoclonal antibody (Mab) fusion with high affinity and specificity is described, conjugated (e.g., expressed) with one or more fusion tag. These fusions can be used, for example, as reagents in diagnostic assays, such as point-of-care (POC) rapid immunoassays and ELISA for in-vitro diagnostic (IVD) testing. Immmunofluorescence-based lateral-flow immunoassays comprise, in one embodiment, one or more of the antibody fusions described herein.

The disclosure further relates to nucleic acids. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. The nucleic acid compositions of the present disclosure, while often in a native sequence (except for modified restriction sites and the like), from either cDNA, genomic or mixtures thereof, may be mutated in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated.

In yet another aspect, a method of producing an antibody fusion is provided, the method comprises (a) obtaining the nucleic acid sequence of an antibody or fragment thereof of interest, (b) operably linking the antibody or fragment thereof nucleic acid to a nucleic acid sequence of a label of interest, (c) expressing the antibody or fragment thereof with the label of interest as an antibody fusion in a host cell and (d) isolating the antibody fusion. The antibody of interest can comprise any antibody. In some embodiments, the antibody of interest can be selected from the group consisting of anti-PCT antibodies, anti-thyroid TRAb, anti-lyme VlsE/C6, anti-OspC/10 and anti-DbpA antibodies. It will be appreciated that additional antibodies, such as those capable of binding to a variety of infectious disease agents, those useful in toxicology and/or allergy panels, allergy panels, as well as antibodies to hormones (e.g., hCG, etc.) are within the scope of the method. The label of interest can be a luminescent label comprising a luciferase and/or a fluorescent label comprising at least one of GFP (green fluorescent proteins), RFP (red fluorescent proteins), CFP (cyan fluorescent proteins), or YFP (yellow fluorescent proteins); and/or a phosphatase label and/or tags such as avidin/biotin or the HaloTag® system sold by Promega® Corp. Madison Wis.).

It will be appreciated that the luciferase can be at least one of NLuc (NanoLuc), RLuc (RetinaLuc), and FLuc (FireflyLuc).

In one embodiment, the phosphatase label comprises SEAP (Secreted Embryonic Alkaline Phosphatase).

In another embodiment, the fluorescent label comprises GFP (green fluorescent proteins), RFP (red fluorescent protein), CFP (cyan fluorescent protein), or YFP (yellow fluorescent protein).

In another embodiment, the antibody of interest comprises an anti-PCT antibody or fragments thereof, and the label of interest comprises NanoLuc.

In yet another embodiment, the antibody of interest comprises an anti-PCT antibody or fragments thereof and the label of interest comprises SEAP.

In still another embodiment, the antibody of interest comprises an anti-thyroid TRAb or fragments thereof and the label of interest comprises NLuc.

In still another embodiment, the antibody of interest comprises a Lyme VlsE/C6 antibody, an OspC/10 antibody, or a DbpA antibody or fragments thereof.

In yet another embodiment, the antibody of interest comprises an M22 (TSHR-specific) antibody or fragments thereof, and the label of interest comprises a fluorescent protein comprising Green Fluorescent Protein (GFP).

In another embodiment, the antibody of interest comprises an M22 (TSHR-specific) antibody or fragments thereof, and the label of interest comprises a fluorescent protein comprising Red Fluorescent Protein (RFP).

In still another embodiment, the antibody of interest comprises M22_NLuc or fragments thereof, and wherein said antibody is paired with a second antibody comprising an RPE-anti human lgG or fragments thereof.

In yet another aspect, an antibody fusion comprising any antibody or fragment thereof and a label of interest selected from the group consisting of (a) a luciferase, (b) a fluorescent protein, and (c) SEAP (Secreted Embryonic Alkaline Phosphatase) is provided. In some embodiments, the antibody of interest is selected from the group consisting of anti-PCT antibodies, anti-thyroid TRAb, anti-lyme VlsE/C6, anti-OspC/10 and anti-DbpA antibodies,

In one embodiment, the antibody of interest is labeled with fusion protein such as avidin or a form of avidin or the HaloTag® protein while the support that the MAb will be attached to will have been labeled with biotin or the small HaloTag linker. Once the MAb comes in contact a covalent or new covalent bonds form ensuring that the MAb is securely attached without being exposed to labeling reagents.

The fluorescent protein can be at least one of GFP (green fluorescent proteins), RFP (red fluorescent protein), CFP (cyan fluorescent protein), or YFP (yellow fluorescent protein).

It will be appreciated that the luciferase can be at least one of NLuc (NanoLuc), RLuc (RetinaLuc), and FLuc (FireflyLuc).

In still another aspect, methods for diagnosing and/or detecting a disease or disorder of interest in a human subject are provided. The method comprises (a) providing an immunoassay comprising an antibody fusion wherein the fusion comprises a label, (b) contacting the immunoassay with a sample from a subject; and (c) detecting whether the antibody fusion binds to a target in the sample to determine presence or absence of said disease or disorder.

It will be appreciated that the label can be SEAP, and/or a luminescent and/or a fluorescent label. The luminescent or fluorescent label can be selected from the group consisting of: a luciferase, GFP (green fluorescent proteins), RFP (red fluorescent protein), CFP (cyan fluorescent protein), or YFP (yellow fluorescent protein).

In one embodiment, the detecting step further can further comprise lateral flow detection.

In another embodiment, the method can further comprise a diagnostic testing system, wherein the diagnostic testing system comprises a lateral flow immunoassay with a fluorescently labelled antibody.

It will be appreciated that the diagnostic testing system can further comprise means to record and display instrument and user history data.

In still another aspect, a plasmid comprising a nucleic acid sequence encoding the antibody fusions described herein is provided.

In another aspect, vectors comprising these plasmids are provided.

In still another aspect, host cells comprising these vectors are provided.

In yet another aspect, kits comprising the antibody fusions described herein are provided.

In other embodiments, the nucleic acids are in operable linkage to another nucleic acid sequence (e.g., the two nucleic acids are in a functional relationship with one another). For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

### DETAILED DESCRIPTION

It will be understood that this disclosure is not limited to particular embodiments described, and as such may vary. A number of various embodiments of the present disclosure are described in detail hereinafter. These embodiments may take many different forms and should not be construed as limited to those embodiments explicitly set forth herein. Rather, these embodiments are provided so that this disclosure will be thorough and complete, and will fully convey the scope of the present disclosure to those skilled in the art. It is also to be understood that the terminology used herein is for the purpose of describing particular embodiments only, and is not intended to be limiting, since the scope of the invention will be limited only by the appended claims.

All patents, applications, published applications and other publications referred to herein are incorporated by reference in their entirety.

The nucleic acid sequences of the disclosure, including fragments thereof (e.g., which encode various CDR regions and/or FR regions, as provided above), can be placed in operable linkage with another nucleic acid, e.g., an empty vector, using routine laboratory techniques and reagents.

Nucleic acid molecules of the present disclosure can be in the form of RNA, such as mRNA, hnRNA, tRNA. pRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand. Furthermore, nucleic acid molecules of the present disclosure which comprise a nucleic acid encoding an antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

The disclosure provides vectors, preferably, expression vectors, containing a nucleic acid encoding the antibody, or may be used to obtain plasmids containing various antibody HC or LC genes or portions thereof. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. The present disclosure also relates to vectors that include isolated nucleic acid molecules of the present disclosure, host cells that are genetically engineered with the recombinant vectors, and the production of at least one antibody by recombinant techniques, as is well known in the art.

For expression of the antibodies, or antibody fragments thereof, DNAs encoding partial or full-length light and heavy chains, can be inserted into expression cassettes or vectors such that the genes are operatively linked to transcriptional and translational control sequences. A cassette which encodes an antibody, can be assembled as a construct. A construct can be prepared using methods known in the art. The construct can be prepared as part of a larger plasmid. Such preparation allows the cloning and selection of the correct constructions in an efficient manner. The construct can be located between convenient restriction sites on the plasmid or other vector so that they can be easily isolated from the remaining plasmid sequences. The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VI, segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (e.g., a signal peptide from a non-immunoglobulin protein).

Although it is possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

In general, a mammalian expression vector contains (1) regulatory elements, usually in the form of viral promoter or enhancer sequences and characterized by a broad host and tissue range; (2) a “polylinker” sequence, facilitating the insertion of a DNA fragment which comprises the antibody coding sequence within the plasmid vector; and (3) the sequences responsible for intron splicing and polyadenylation of mRNA transcripts. This contiguous region of the promoter-polylinker-polyadenylation site is commonly referred to as the transcription unit. The vector will likely also contain (4) a selectable marker gene(s) (e.g., the beta-lactamase gene), often conferring resistance to an antibiotic (such as ampicillin), allowing selection of initial positive transformants in E. coli; and (5) sequences facilitating the replication of the vector in both bacterial and mammalian hosts.

Alternatively, the nucleic acids encoding the antibody sequence can be expressed in stable cell lines that contain the gene integrated into a chromosome. The co-transfection with a selectable marker such as DHFR, GPT, neomycin, or hygromycin allows the identification and isolation of the transfected cells which express large amounts of the encoded antibody.

Examples of suitable inducible non-fusion E. coli expression vectors include pTrc and pET 11d. Examples of vectors for expression in yeast S. cerevisiae include pYepSec1, pMFa, pJRY88, pYES2, and pPicZ (Invitrogen Corp, San Diego, Calif., USA). Examples of baculovirus vectors available for expression of proteins in cultured insect cells (e.g., Sf 9 or Hi5 cells) include the pOET, pTriEx, plEx, pBAC, pBacPAK, and the BD pVL and pAc families of vectors (Expression Systems LLC, Davis, Calif., USA).

In yet another embodiment, a nucleic acid of the disclosure is expressed in mammalian cells using a mammalian expression vector. Examples of mammalian expression vectors include pCDM8 (Seed et al., Nature, 329:840, 1987) and pMT2PC (Kaufman et al., EMBO J, 6:187-195, 1987). Preferably, the recombinant mammalian expression vector is capable of directing expression of the nucleic acid, preferentially in a particular cell type, such as lymphoma cells (e.g., mouse myeloma cells). In specific cell types, tissue-specific regulatory elements are used to express the nucleic acid. Tissue-specific regulatory elements are known in the art.

The disclosure further provides a recombinant expression vector comprising a DNA molecule cloned into the expression vector in an antisense orientation. That is, the DNA molecule is operably linked to a regulatory sequence in a manner that allows for expression (by transcription of the DNA molecule) of an RNA molecule that is antisense to the mRNA encoding a polypeptide. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. See Weintraub et al., Reviews—Trends in Genetics, 1, 1986).

In some embodiments, the nucleic acids encoding the binding agents (such as antibodies) of the disclosure are transfected in mammalian cells such as CHO cells, myeloma cells, HEK293 cells, BHK cells (BHK21, ATCC CRL-10), mouse Ltk-cells, COS cells, and NIH3T3 cells have been frequently used for stable expression of heterologous genes. In an alternative method of producing the antibodies of the disclosure, a non-human animal in which is one or more, and preferably essentially all, of the cells of the animal contain a heterologous nucleic acid introduced by way of human intervention, a transgene, coding for the antibody. The transgene can be introduced into the cell, directly or indirectly, by introduction into a precursor of the cell, by way of deliberate genetic manipulation, such as by microinjection or by infection with a recombinant virus. Methods for generating non-human transgenic mammals are known in the art. See, e.g., but not limited to, U.S. Pat. Nos. 5,827,690; 5,849,992; 4,873,316; 5,849,992; 5,994,616; 5,565,362; and 5,304,489. Such methods can involve introducing DNA constructs into the germ line of a mammal to make a transgenic mammal. Methods of producing transgenic animals using a somatic cell are described in U.S. Pat. No. 6,147,276; Baguisi et al. Nature Biotech., 17, 456-461, 1999; Campbell et al., Nature, 380, 64-66, 1996; Cibelli et al., Science, 280, 1256-8, 1998; Kato et al., Science, 282, 2095-2098, 1998; Schnieke et al., Science, 278, 2130-2133, 1997; Wakayama et al., Nature, 394, 369-374, 1998.

The antibodies may be produced in mammary glands of animals using promoters that are preferentially activated in mammary epithelial cells, including promoters that control the genes encoding milk proteins such as caseins. See, Clark et al., Bio Technology, 7: 487-492, 1989; Gordon et al. Bio Technology, 5: 1183-1187, 1987). Binding agents (such as antibodies) of the present disclosure can additionally be produced using at least one antibody encoding nucleic acid to provide transgenic plants and cultured plant cells (e.g., tobacco, maize, and duckweed). See, Cramer et al., Curr. Top. Microbol. Immun., 240:95-118, 1999.

The nucleic acids of the present disclosure can also be prepared by direct chemical synthesis by known methods, e.g., U.S. Pat. Nos. 5,942,609; 6,521,427; 6,586,211; and 6,670,127.

Once prepared, the antibody fusion can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography such as with a Protein A column, hydroxylapatite chromatography, lectin chromatography, HPLC, and the like. Antibodies of the present disclosure include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present disclosure can be glycosylated or can be non-glycosylated, with glycosylated preferred.

Amino acids in antibodies of the present disclosure that are important for function, e.g., binding, can be identified by methods known in the art, such as site-directed mutagenesis or alanine-scanning mutagenesis (e.g., Ausubel, Current Protocols (2002), Chapters 8, 15, supra; Cunningham et al., Science 244:1081-1085, 1989). Cunningham's procedure introduces single alanine mutations at every residue in the molecule. The resulting mutant molecules are then tested for biological activity, such as binding activity. Sites that are important for antibody binding can also be identified by structural analysis such as crystallization, nuclear magnetic resonance or photo affinity labeling (Smith et al., J. Mol. Biol., 224:899-904, 1992 and de Vos et al., Science 255:306-312, 1992).

### Generation of Antibody Fusions

Antibody fusions of the present disclosure can be optionally produced by a variety of techniques. The antibody itself can be optionally generated by hybridoma techniques, or for example, immunization of a transgenic animal (e.g., mouse, rat, hamster, non-human primate, and the like) capable of producing a repertoire of human antibodies, as described herein.

The use of transgenic mice carrying human immunoglobulin (Ig) loci in their germline configuration provide for the isolation of high affinity fully human monoclonal antibodies directed against a variety of targets including human self antigens for which the normal human immune system is tolerant (See, Lonberg et al., Nature, 368, 856-9, 1994; Green et al., Nature Genet., 7, 13-21, 1994; Green et al., Exp. Med., 188:483-95, 1988; Lonberg et al., Int. Rev. Immunol., 13:65-93, 1995; Bruggemann et al., Eur. J. Immunol., 21, 1323-1326, 1991; Fishwild et al., Nat. Biotechnol., 14:845-851, 1996; Mendez et al., Nat. Genet., 15:146-156, 1997; Green et al., J. Immunol. Methods 231:11-23, 1999; Yang et al., Cancer Res. 59:1236-1243, 1999; Brüggemann et al., Curr. Opin. Biotechnol. 8:455-458, 1997; and U.S. Pat. Nos. 5,569,825; 6,300,129; 6,713,610; 7,041,870). The endogenous immunoglobulin loci in such mice can be disrupted or deleted to eliminate the capacity of the animal to produce antibodies encoded by endogenous genes. In addition, companies such as Codexis, Inc. (Redwood City, Calif., USA) and Creative Biolabs, Inc. (Shirley, N.Y., USA) can be engaged to provide human antibodies directed against a selected antigen using technology as described above.

Preparation of immunogenic antigens, and monoclonal antibody production can be performed using any suitable technique such as recombinant methods. The immunogenic antigens can be administered to an animal in the form of purified form or synthetic form. At least two forms are described in the Examples.

Immunization with antigen can be optionally accompanied by addition of an adjuvant, such as complete Freund's adjuvant. The immune response can be monitored over the course of the immunization protocol with plasma samples being obtained by retroorbital bleeds. The plasma can be screened by ELISA (as described below), and animals, e.g., rabbits or mice, with sufficient titers of immunoglobulin can be used for fusions. Animals can be boosted intravenously with antigen 3 days before sacrifice and removal of the spleen. In some embodiments, plurality (e.g., 2, 3, 4 or more) of antigen fusions may be performed. Several animals may be immunized for each antigen.

To generate hybridomas producing monoclonal antibodies, splenocytes and lymph node cells from immunized animals can be isolated and fused to an appropriate immortalized cell line, such as a mouse myeloma cell line. The resulting hybridomas can be screened for the production of antigen-specific antibodies.

A suitable immortal cell line incapable of producing immunoglobulin chains is selected as a fusion partner, e.g., a myeloma cell line such as, but not limited to, Sp2/0 and derivative cell lines, NS1 and derivatives, especially NSO engineered NSO lines such as GS-NSO, AE-1, L.5, P3X63Ag8.653, U937, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O or the like, or hetero-myelomas, fusion products thereof, or any cell or fusion cell derived therefrom, or any other suitable cell line as known in the art (Birch et al., Biologics 22:127-133, 1994). The fused cells (hybridomas) or recombinant cells can be isolated using selective culture conditions or other suitable known methods, and cloned by limiting dilution or cell sorting, or other known methods. Cells which produce antibodies with the desired specificity can be detected by a suitable assay (e.g., ELISA) and selected for manipulation.

Other suitable methods of generating or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; Morphosys®, Martinsreid, Germany; Biovation, Aberdeen, Scotland, UK; Biolnvent, Lund, Sweden; Dyax Corp., Enzon, Affymax/Biosite; Xoma, Berkeley, Calif., USA. See, e.g., U.S. Pat. Nos. 5,885,793; 5,969,108; 5,994,519; 6,017,732; 6,248,516; or stochastically generated peptides or proteins (U.S. Pat. Nos. 5,723,323; 5,763,192; 5,814,476; 5,817,483; 5,824,514; 5,976,862) that are capable of producing a repertoire of human antibodies, as known in the art and/or as described herein. Such techniques, include, but are not limited to, ribosome display (Hanes et al., PNAS USA, 94:4937-4942, 1997); Hanes et al., PNAS USA, 95:14130-1413, 1998); single cell antibody producing technologies (e.g., selected lymphocyte antibody method (“SLAM”) (U.S. Pat. No. 5,627,052; Wen et al., J. Immunol., 17:887-892, 1987; Babcook et al., PNAS USA, 93:7843-7848, 1996); gel microdroplet and flow cytometry (Powell et al., Biotechnol., 8:333-337, 1990; One Cell Systems, Cambridge, Mass., USA; Gray et al., J Imm. Meth., 182:155-163, 1995; Kenny et al., Bio Technol., 13:787-790, 1995); B-cell selection (Steenbakkers et al., Molec. Biol. Reports, 19:125-134, 1994; Jonak et al., Progress Biotech., Vol. 5, In vitro Immunization in Hybridoma Technology, Borrebaeck, ed., Elsevier, Amsterdam, Netherlands, 1988).

Also included are kits comprising the antibody or an antigen-binding fragment thereof as provided in the foregoing or following paragraphs and instructions for using the kits, e.g., in diagnosis of diseases, disorders, infections, or conditions.

Screening antibodies for specific binding to similar proteins or fragments can also be conveniently achieved using peptide display libraries. This method involves the screening of large collections of peptides for individual members having the desired function or structure. Antibody screening using peptide display libraries is well known in the art. The displayed peptide sequences can be from 3 to 5000 or more amino acids in length, frequently from 5-100 amino acids long, and often from about 8 to 25 amino acids long. Peptide display libraries, vector, and screening kits are commercially available from such suppliers as Invitrogen (Carlsbad, Calif., USA), and Cambridge Antibody Technologies (Cambridgeshire, UK). See, U.S. Pat. No. 5,885,793. See also, e.g., Enzon patents (U.S. Pat. Nos. 4,704,692; 4,939,666; 4,946,778; 5,260,203; 5,455,030; 5,518,889; 5,534,621; 5,656,730; 5,763,733; 5,767,260; and 5,856,456); Dyax patents (U.S. Pat. Nos. 5,223,409; 5,403,484; 5,571,698; and 5,837,500); Affymax patents (U.S. Pat. Nos. 5,427,908; 5,580,717); Genentech patents (U.S. Pat. No. 5,750,373); and Xoma patents (U.S. Pat. Nos. 5,618,920; 5,595,898; 5,576,195; 5,698,435; and 5,693,493; 5,698,417).

### Antibody Fragments

Antibody fragments can be derived via proteolytic digestion of intact antibodies (see, e.g., Morimoto et al., J Biochem Biophys Methods, 24:107-117, 1992; and Brennan et al., Science, 229:81, 1985). However, these fragments can now be produced directly by recombinant host cells. F(ab′)2, Fab, Fv and ScFv antibody fragments can all be expressed in and secreted from mammalian host cells or from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., BioTechnology 10:163-167, 1992).

Preferably, recombinant production of antibody fragments is carried out using a single-chain expression polynucleotide. This expression polynucleotide contains: (1) a single-chain antibody cassette consisting of a VH domain, spacer peptide, and VL domain operably linked to encode a single-chain antibody, (2) a promoter suitable for in vitro transcription (e.g., T7 promoter, SP6 promoter, and the like) operably linked to ensure in vitro transcription of the single-chain antibody cassette forming a mRNA encoding a single-chain antibody, and (3) a transcription termination sequence suitable for functioning in an in vitro transcription reaction. Optionally, the expression polynucleotide may also comprise an origin of replication and/or a selectable marker. An example of a suitable expression polynucleotide is pLM166. To obtain VH and VL sequences for cloning, a library of VH and VL sequences produced by PCR amplification using V gene family-specific primers or V gene-specific primers may be used (Nicholls et al., J. Immunol. Meth., 165: 81, 1993; WO 1993/12227) or are designed according to standard art-known methods based on available sequence information. Typically, mouse or human VH and VL sequences are isolated. The VH and VL sequences are then ligated, usually with an intervening spacer sequence (e.g., encoding an in-frame flexible peptide spacer), forming a cassette encoding a single-chain antibody. Typically, a library comprising a plurality of VH and VL sequences is used (sometimes also with a plurality of spacer peptide species represented), wherein the library is constructed with one or more of the VH and VL sequences mutated to increase sequence diversity particularly at CDR residues, sometimes at framework residues. V region sequences can be conveniently cloned as cDNAs or PCR amplification products for immunoglobulin-expressing cells. For example, cells from human hybridoma, or lymphoma, or other cell line that synthesizes either cell surface or secreted immunoglobulin may be used for the isolation of polyA+RNA. The RNA is then used for the synthesis of oligo dT primed cDNA using the enzyme reverse transcriptase (see, Goodspeed et al., Gene, 76: 1, 1989; Dunn et al., J. Biol. Chem., 264: 13057, 1989). Once the V-region cDNA or PCR product is isolated, it is cloned into a vector to form a single-chain antibody cassette.

In some embodiments, the antibodies of the disclosure or antigen-binding fragments thereof may be prepared by in vitro (e.g., cell-free) synthesis, using conventional methods as known in the art. Various synthetic apparatuses are available, e.g., automated synthesizers by Applied Biosystems, Inc., Foster City, Calif., USA. By using synthesizers, naturally occurring amino acids may be substituted with unnatural amino acids. The particular sequence and the manner of preparation will be determined by convenience, economics, purity required, and the like.

In some embodiments, the engineered antibody molecules of the disclosure may contain a peptide sequence, for example, an N-terminal signal sequence that guides the trafficking of the antibody or a fragment thereof to the extracellular milieu, plasma membrane (outer membrane, transmembrane, or inner membrane), or a specialized compartment in the cell, e.g., endosome, lysosome, ER, Golgi's apparatus, vacuoles, inclusion bodies, nucleolus, mitochondria, chloroplast, periplasm, etc.

Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5, 256-262, 1993 and Plückthun et al., Immunol. Revs., 130:151-188, 1992.

Known methods of DNA or RNA amplification include, but are not limited to, polymerase chain reaction (PCR) and related amplification processes. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in U.S. Pat. No. 4,683,202. Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

The isolated nucleic acid compositions of this disclosure, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries, is well known to those of ordinary skill in the art.

In some embodiments, mutations can be introduced randomly along all or part of an antibody coding sequence, such as by saturation mutagenesis or by recombination, and the resulting modified antibodies can be screened for binding activity.

The addition, removal or modification of the constant regions of the antibody is known to play a particularly important role in the bioavailability, distribution, and half-life of therapeutically administered antibodies. The antibody class and subclass, encoded by the Fc or constant region of the antibody, when present, imparts important additional properties. Thus, antibodies with reconfigured, redesigned, or otherwise altered constant domains are encompassed by the antibody compositions of the disclosure.

The disclosure further relates to compositions comprising at least one antibody fusion and a carrier. Preferably the composition is a pharmaceutical composition comprising at least one antibody fusion and a pharmaceutically acceptable carrier. The compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Remington's Pharmaceutical Sciences, Gennaro et al., Ed., 18^{th}Edition, Mack Publishing Co., Easton, Pa., USA (1990). Pharmaceutically acceptable carriers can be routinely selected that are suitable for the mode of administration, solubility and/or stability of the antibody, fragment or variant composition as well known in the art or as described herein.

The disclosure further relates to stable formulations containing the antibody fusions and buffering components and, optionally, stabilizers or preservatives, as well as multi-use formulations suitable for research, diagnostic and/or medical use. Antibody compositions may include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; TRIS, tromethamine hydrochloride, or phosphate buffers. Preferred buffers for use in the present compositions are amino acids or organic acid salts such as citrate. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine. The formulations can cover a wide range of pHs, such as from about pH 4 to about pH 10, and preferred ranges from about pH 5 to about pH 9, and a most preferred range of about 6.0 to about 8.0. Preferably the formulations of the present disclosure have pH between about 6.8 and about 7.8.

Embodiments of the disclosure further provide for surfaces comprising the aforementioned antibody or antigen compositions, wherein the antibody or antigen is oriented to permit binding to a partner. Preferably, the surface is a surface of a solid support. Numerous and varied solid supports are known to those in the art and include, without limitation, nitrocellulose, the walls of wells of a reaction tray, multi-well plates, test tubes, polystyrene beads, magnetic beads, membranes, and microparticles (such as latex particles). Nitrocellulose, nylon and other microporous structures are useful, as are materials with gel structure in the hydrated state. Further examples of useful solid supports include natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers and the like.

Preferably, the support is a well of an array plate, e.g., a microarray such as a protein array or an antibody array. Methods for constructing such arrays are known in the art.

There are many solid supports which can be used for the method and the kit of the present disclosure. Well known materials which may be employed include glass, polystyrene, polypropylenes dextran, nylon, agarose, dextran, acrylamide, nitrocellulose, PVDF and other materials, in the form of tubes, beads, membranes and microtiter plates formed from or coated with such materials, and the like. The isolated and purified recombinant polypeptides and/or the antibodies of the present disclosure can be either covalently or physically bound to the solid support, by techniques such as covalent bonding via an amide, ester or disulfide linkage, or by adsorption. This binding or immobilization can be accomplished by using e.g., covalent bonding via an amide, ester or disulfide linkage between the solid support and the antibodies (e.g., via the Fc domain) or the epitopes of the antigen. Active linkers, such as avidin and/or biotin, or HaloTag® (which includes a fusion protein and a small tag (a non-protein molecule similar in size to biotin can also be used in accordance with the invention. In case the antigen/antibody is fused to GST, the fusion polypeptide is preferably immobilized in such a way that it is aligned on the solid support via a disulfide linkage between the solid support presenting glutathione on its surface and the GST portion of the polypeptide. Presently preferred for use as a solid support are micro titer plates made of polystyrole which can be obtained from various commercial suppliers such as NUNC, Costar, or Greiner.

In case the method of detecting and/or quantifying antigens is performed on a solid support, usually, the solid support is coated with the isolated and purified recombinant antibodies or antigen-binding fragments thereof. Coating may be performed by using a coating buffer known to the person skilled in the art such as PBS buffer or carbonate buffer. Such coating buffers as well as solid supports already coated might be included as reagents to the kit of the present disclosure. In a preferred mode for performing the above-described method of the disclosure it is important to use certain “blockers” which might be included as a reagent in the kit of the disclosure as well. The “blockers” are added to assure that non-specific proteins, protease, or antibodies other than those that bind specifically to the antigenic peptides do not cross-link or destroy the antigens or antibodies on the solid support, or the radiolabeled indicator antigen or antibody, to yield false positive or false negative results. A usual blocker which can be used is bovine serum albumin (BSA), which is preferred. The blocker can be added in buffer solution like PBS buffer. In case a solid support is used the blocker is usually added after coating the solid support.

The disclosure further relates to compositions or kits comprising the immunogens of the disclosure. The composition can comprise, in addition to the immunogen, one or more of: a salt; a solubilizing agent; a detergent, e.g., a non-ionic detergent such as TWEEN-20, etc.; a protease inhibitor; glycerol; and the like.

Compositions comprising the immunogen may include a buffer, which is selected according to the desired use of the peptide, and may also include other substances appropriate to the intended use. Those skilled in the art can readily select an appropriate buffer, a wide variety of which is known.

In some embodiments, a composition comprising the immunogen is a diagnostic composition. Diagnostic compositions according to the disclosure can, for example, be employed in usual immunoassays in which the at least one peptide of the composition is reacted with antibodies of the disclosure (as controls). As stated above the disclosure does not only cover diagnostic compositions but also especially immunoassay methods in which the compositions are used as antigenic substance. For heterogeneous assays, the immunogen may be linked to solid support. Independent on the form of the assay, a tracer complex composed of at least one immunogen linked to a marker, e.g., a fluorescent or a luminescent molecule, either directly or via a linker, may be used. Such reagents are especially useful in SPR assays.

Exemplary linkers include, e.g., glycine linkers, such as, single or oligomeric glycine (e.g., G, GG, GGG or the like), glycine polymers (G)n, (e.g., where n is an integer from 1 to about 20); glycine-serine polymers (including, for example, (GS)n, (GSGGS)n (SEQ ID NO: 1) and (GGGS)n (SEQ ID NO: 2), where n is an integer of between 1 and 10, e.g., 1, 2, 3, 4, 5, 6, 7, or more; preferably 1, 2 or 3), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are used in some embodiments. Exemplary flexible linkers include, but are not limited to G, GG, GGG, GGGG (SEQ ID NO: 3), GGGGG (SEQ ID NO: 4), GGS, GGSG(SEQ ID NO: 5), GGSGG(SEQ ID NO: 6), GSGSG (SEQ ID NO: 7), GSGGG (SEQ ID NO: 8), GGGSG (SEQ ID NO: 9), GSSSG (SEQ ID NO: 10), and the like.

The disclosure further relates to kits or other articles of manufacture which contains one or more immunogens or a composition comprising the same, together with instructions for formulating and/or using the composition, e.g., generating antibodies. Kits or other articles of manufacture may include a container, a syringe, vial, a surface, or any other article, device or equipment useful in conducting the diagnostic test (e.g., in vitro or ex vivo). Diagnostic tests may also be conducted in vivo. Suitable containers include, for example, bottles, vials, syringes (e.g., pre-filled syringes), ampules, cartridges, reservoirs, pumps, or lyo-jects. The container may be formed from a variety of materials such as glass or plastic.

Compositions and/or kits for manufacture of the polypeptide may include whole cells and a carrier, e.g., buffer. Embodiments of the instant disclosure further provide for systems, e.g., diagnostic systems or immunoapheresis systems, comprising the aforementioned compositions and/or kits.

The disclosure further includes nucleic acids encoding the peptide components of the peptidoglycan immunogens of the disclosure, including vectors comprising said nucleic acids, and cells comprising such nucleic acids and/or vectors.

The disclosure is directed inter alia to the detection of antigens that are diagnostic of diseases or disorders. The disclosure provides specific and sensitive assays for diagnosing such diseases, thereby providing clarity to clinical assessment of the patient.

One aspect of the disclosure is a method for detecting and/or diagnosing diseases or disorders in a subject suspected of having antibody against a causative agent of the disease or disorder. The diagnostic method is useful for diagnosing subjects exhibiting the clinical symptoms of, or suspected of having, the disease or disorder. In another aspect of the disclosure, there is provided a method for detecting a disease or disorder in the subject comprising detecting a desired antigen using one or more antibody fusions of the disclosure.

Preferably, the disclosure provides a method for diagnosing a disease or disorder in a subject comprising measuring a bodily fluid of the subject for the presence of an antigen of interest, wherein an elevated level of the antigen in the subject compared to a corresponding level of antibody in a control (such as a known unaffected subject) indicates an infection by the causative agent and/or that the subject has the a disease or disorder.

One embodiment of this method comprises contacting (incubating, reacting) a sample of a biological fluid (e.g., urine, serum, whole blood or CSF) from a subject to be diagnosed (a subject suspected of having a disease or disorder with the diagnostic reagent comprising the antibody fusions of the disclosure. In the presence of an antibody response to infection, an antigen-antibody complex is formed. Subsequently the reaction mixture is analyzed to determine the presence or absence of this antigen-antibody complex. A variety of conventional assay formats can be employed for the detection, such, e.g., as ELISA, microarray analysis, Luminex bead based assays or lateral flow methods. The presence of an elevated amount of the antibody-peptide complex indicates that the subject was exposed to and infected with a pathogen. In any detection assay of the disclosure, a positive response is defined as a value of 1.5, 2, 3, 4 or more, e.g., 5 standard deviations greater than the mean value of a group of healthy controls. For the purposes of the initial screening, a positive response is defined as a statistically significant difference in the mean binding of diagnostic reagent compared to controls (e.g., a healthy subject). Statistical significance may be determined using routine statistical tests.

One embodiment of the disclosure is a diagnostic immunoassay method, which comprises (1) taking a sample of body fluid or tissue likely to contain immunogens or antibodies thereto; (2) contacting the sample with an antibody of the disclosure or a peptide of the disclosure, under conditions effective for the formation of a specific antibody-antigen complex, e.g., reacting or incubating the sample and the antibody of the disclosure (or reacting or incubating the sample and the immunogen); and (3) assaying the contacted (reacted) sample for the presence of an antibody-antigen complex (e.g., determining the amount of an antibody-peptide complex).

Conditions for reacting peptides and antibodies so that they react specifically are well-known to those of skill in the art. See, e.g., Current Protocols in Immunology, Coligan et al, Eds., John Wiley & Sons, Inc., N.Y. (2003) or the Examples herein.

The sample is preferably easy to obtain and may be serum or plasma derived from a venous blood sample or even from a finger prick. Tissue from other body parts or other bodily fluids, such as cerebro-spinal fluid (CSF), saliva (oral fluid), sputum, phlegm, nasal discharge, mucus, tear, tric secretions, etc. are known to contain antigens (or antibodies thereto) and may be used as a source of the sample.

Once the analyte and the probe are permitted to react in a suitable medium, an assay is performed to determine the presence or absence of an antibody-peptide reaction. Among the many types of suitable assays, which will be evident to a skilled worker, are ELISA, immunoprecipitation and agglutination assays.

The protocols for immunoassays using antigens for detection of specific antibodies are well known in art. For example, a conventional sandwich assay can be used, or a conventional competitive assay format can be used. For a discussion of some suitable types of assays, see Current Protocols in Immunology, supra). In a preferred assay, an antibody of the disclosure is immobilized to the solid or semi-solid surface or carrier by means of covalent or non-covalent binding, either prior to or after the addition of the sample comprising or believed to contain an antigen (e.g., or a variant thereof).

Devices for performing specific binding assays, especially immunoassays, are known and can be readily adapted for use in the present methods. Solid phase assays, in general, are easier to perform than heterogeneous assay methods which require a separation step, such as precipitation, centrifugation, filtration, chromatography, or magnetism, because separation of reagents is faster and simpler. Solid-phase assay devices include microtiter plates, flow-through assay devices, dipsticks and immunocapillary or immunochromatographic immunoassay devices.

In embodiments of the disclosure, the solid or semi-solid surface or carrier is the floor or wall in a microtiter well; a filter surface or membrane (e.g. a nitrocellulose membrane or a PVDF (polyvinylidene fluoride) membrane, such as an Immobilon membrane); a hollow fiber; a beaded chromatographic medium (e.g. an agarose or polyacrylamide gel); a magnetic bead; a fibrous cellulose matrix; an HPLC matrix; an FPLC matrix; a substance having molecules of such a size that the molecules with the peptide bound thereto, when dissolved or dispersed in a liquid phase, can be retained by means of a filter; a substance capable of forming micelles or participating in the formation of micelles allowing a liquid phase to be changed or exchanged without entraining the micelles; a water-soluble polymer; or any other suitable carrier, support or surface.

In embodiments of the disclosure, the detection procedure comprises visibly inspecting the antibody-peptide complex for a color change, or inspecting the antibody-peptide complex for a physical-chemical change. Physical-chemical changes may occur with oxidation reactions or other chemical reactions. They may be detected by eye, using a spectrophotometer, or the like.

In one embodiment of the method, the probe is electro- or dot-blotted onto nitrocellulose paper. Subsequently, the biological fluid (e.g., serum or plasma) is incubated with the blotted probe, and analyte in the biological fluid is allowed to bind to the probe(s). The bound complex can then be detected, e.g. by standard immunoenzymatic methods. In another embodiment of the method, latex or polystyrene beads are conjugated to the probes and the biological fluid is incubated with the bead/probe conjugate, thereby forming a reaction mixture. The reaction mixture is then analyzed to determine the presence of the analyte.

One assay for the screening of blood products or other physiological or biological fluids is ELISA. Typically in an ELISA, the probe of the disclosure is adsorbed to the surface of a microtiter well directly or through a capture matrix. Residual, non-specific protein-binding sites on the surface are then blocked with an appropriate agent, such as BSA, heat-inactivated normal goat serum (NGS), or BLOTTO (a buffered solution of nonfat dry milk). The well is then incubated with a biological sample suspected of containing pathogenic analyte. The sample can be applied neatly, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound analyte and then incubated with an optimal concentration of an appropriate anti-immunoglobulin antibody (e.g., for human subjects, an anti-human immunoglobulin (ctHuIg) from another animal, such as dog, mouse, cow, etc.) that is conjugated to an enzyme or other label by standard procedures and is dissolved in blocking buffer. The label can be chosen from a variety of enzymes, including horseradish peroxidase (HRP), β-galactosidase, alkaline phosphatase, glucose oxidase, etc. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length).

Another useful assay format is a lateral flow format. Antibody to human or animal antibody or staph A or G protein antibodies is labeled with a signal generator or reporter (i.e., colloidal gold) that is dried and placed on a glass fiber pad (sample application pad). The diagnostic probe is immobilized on membrane, such as a PVDF (polyvinylidene fluoride) membrane (e.g., an IMMOBILON membrane (Millipore)) or a nitrocellulose membrane. When a solution of sample (blood, serum, etc.) is applied to the sample application pad, it dissolves the colloidal gold labeled reporter and this binds to all analyte in the sample. This mixture is transported into the next membrane (PVDF or nitrocellulose containing the diagnostic probe) by capillary action. If the analyte is present in the sample, they bind to the probe striped on the membrane generating a signal. An additional antibody specific to the colloidal gold labeled antibody (such as goat anti-mouse IgG) may be used to produce a control signal.

### BRIEF DESCRIPTION OF THE FIGURES

FIG. 1 shows a schematic representation of the Nanoluc® luciferase labeling system.

FIG. 2 shows M22_NLuc_HisTag antibody protein testing using SD S-PAGE analysis of purified protein with a Ni-NTA column.

FIG. 3A shows M22_NLuc enzyme titration and luminescence activity with furimazine as the substrate.

FIG. 3B shows the L1-10 based ELISA assay results with M22_NLuc titration curve in a 30-minute assay.

FIG. 4 shows the L1-10 based ELISA assay results on an SA microplate with an M22_NLuc titration turve.

FIG. 5 shows the results of an L1-10 based ELISA Assay on SA beads (M22_NLuc Titration Curve).

FIG. 6 shows the M22_NLuc antibody dose response on L1-10 using an anti-MBP or anti-StrepMab) RAM coated ELISA.

FIG. 7 shows the results of the L1-10 based ELISA Assay using a-MBP as capture (M22_NLuc Titration Curve).

FIG. 8 shows M22 dose response with M22_Luc at 120 ng/ml and 40 ng/ml. Results are set forth in in RLU.

FIG. 9 shows M22 dose response with M22_Luc at 30 ng/ml and 10 ng/ml. Results are set forth in in RLU.

FIG. 10 shows SDS-PAGE analysis of purified protein by Ni-NTA column. Protein characterization results demonstrated that the L1-10 fusion protein was purified as 92 kDa on both reduced and non-reduced SDS-PAGE, which was as expected. The purified protein showed as one major dominant peak on HPLC as well.

FIG. 11 shows L110_NLuc Enzyme Titration-Luminescence Activity.

FIG. 12 shows M22 based ELISA Assay-L1-10_NLuc Titration Curve.

FIG. 13 shows L1-10_NLuc 1-step ELISA Assay-M22 Dose Response Curve.

FIG. 14A shows M22 dose response in the L1-10_Nluc (200 ng/ml ) ELISA.

FIG. 14B shows M22 dose response in the L1-10_Nluc (100 ng/ml ) ELISA.

FIG. 15 shows the amino acid sequence of the M22 heavy chain (HC) and light chain (LC), amino acid sequence of the G3 S linker, and the amino acid sequences for GFP protein and His tag.

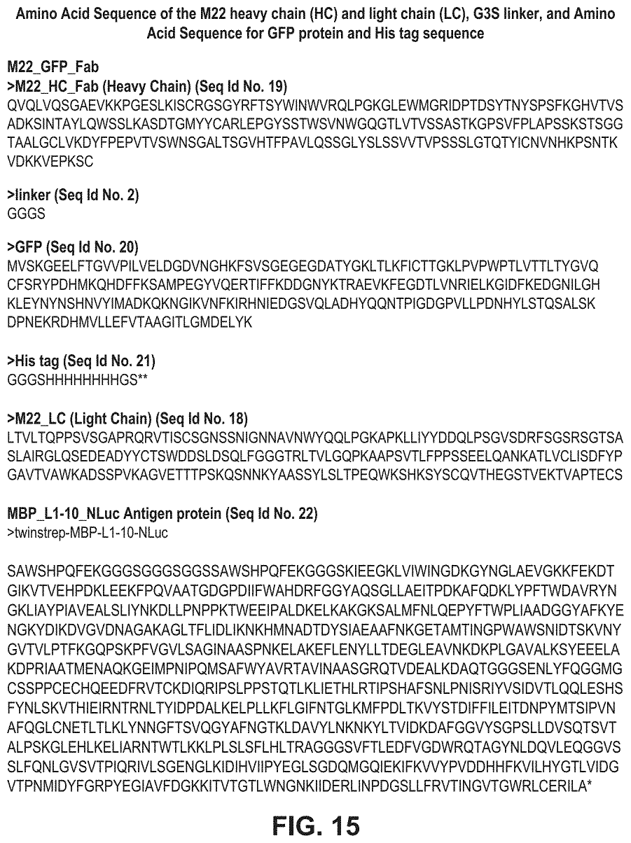

FIG. 16 is a diagrammatical representation of the schema used for the cell surface fluorescence detection of TSHR using the M22-GFP_Fab fusions.

FIG. 17 sets forth the M22-GFP Fab protein expression & purification results via SDS-PAGE analysis of purified protein using a Ni-NTA column.

FIG. 18 sets forth a histogram of M22+ secondary mab results (measured as fluorescence per cell) as well as a dose response curve of the M22 antibody.

FIG. 19 shows M22-GFP fusion fluorescence intensity, measured per cell.

FIG. 20A sets forth a schematic diagram of an antibody fusion of the present technology wherein anti Flu A antibody is fused to a Halo Tag (Promega) protein tag and conjugated to europium beads coupled to ligand. FIG. 20B sets forth a lateral flow immunoassay performed with antibody fusions of the present technology.

### DEFINITIONS

As used herein, the singular forms “a,” “an,” and “the” include plural referents unless the context clearly dictates otherwise. Thus, for example, reference to “a protein” includes a plurality of such proteins and reference to “the formulation” includes reference to one or more formulations and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed by this disclosure. The upper and lower limits of these smaller ranges may independently be included or excluded in the range, and each range where either, neither or both limits are included in the smaller ranges is also encompassed by this disclosure, subject to any specifically excluded limit in the stated range. Where the stated range includes one or both of the limits, ranges excluding either or both of those included limits are also within the scope of this disclosure.

The word “about” means a range of plus or minus 10% of that value, e.g., “about 5” means 4.5 to 5.5, “about 100” means 90 to 100, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about.”

A “binding agent” is a binding fragment of the antibodies described herein. For example, the binding agent can be a full-length antibody (e.g., having an intact variable and constant (Fc) region or an antibody binding fragment (e.g., a Fab, Fab′ or F(ab′)₂, FV or dAb). These antibodies or fragments thereof can be rabbit, rodent, human etc. The binding agent can be single-domain antibodies such as rabbit, camelid or human single VH or VL domains that bind to CWPS. It will be appreciated that binding agents in accordance with the invention can further include proteins, polypeptides, and the like that comprise one or more of the CDRs described herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within 10%, or within 5% or less, e.g., within 2%.

As used herein, the term “plurality” can be 2, 3, 4, 5, 6, 7, 8, 9, 10, or more.

As used herein, “isolated” means a nucleic acid sequence or a polypeptide sequence that is separated from the wild or native sequence in which it naturally occurs or is in an environment different from that in which the sequence naturally occurs.

“Protein,” “polypeptide,” “oligopeptide,” and “peptide” are used interchangeably to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

The term “sequence identity” means nucleic acid or amino acid sequence identity in two or more aligned sequences, aligned using a sequence alignment program. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at (ncbi.nlm.gov/BLAST/). See, also, Altschul, S. F. et al., 1990 and Altschul, S. F. et al., 1997.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 3389-3402, respectively. Software for performing BLAST analyses is publicly available through the National Center for Biotechnology Information website. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

While all of the above mentioned algorithms and programs are suitable for a determination of sequence alignment and % sequence identity, for purposes of the disclosure herein, determination of % sequence identity will typically be performed using the BESTFIT or GAP programs in the GCG Wisconsin Software package (Accelrys, Madison Wis.), using default parameters provided.

The phrase “% sequence identity,” “percent identity,” or “percent identical” refers to the level of nucleic acid or amino acid sequence identity between two or more aligned sequences, when aligned using a sequence alignment program. For example, 70% homology means the same thing as 70% sequence identity determined by a defined algorithm, and accordingly a homologue of a given sequence has greater than 70% sequence identity over a length of the given sequence. Exemplary levels of sequence identity include, but are not limited to 70%, 75% 80%, 85%, 90% or 95% or more sequence identity to a given sequence.

As used herein, the term “synthetic” refers to a molecule, e.g., a polypeptide or a polynucleotide, which has been manufactured by artificial chemical synthesis or biosynthesis (e.g., genetic engineering-based production). Preferably, the term relates to non-naturally-occurring molecules constructed by one of the methods mentioned above or by other suitable methods known in the art.

“Associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

As used herein, the term “amino acid” includes the 22 amino acids that are proteinogenic amino acids and non-proteinogenic amino acids. The term “proteinogenic amino acid,” is used in the field of biochemistry to refer to the 22 amino acids that are incorporated into eukaryotic and/or prokaryotic proteins during translation, such as: (a) histidine (His; H); (b) isoleucine (Ile; I); (c) leucine (Leu; L); (d) Lysine (Lys; K); (e) methionine (Met; M); (f) phenylalanine (Phe; F); (g) threonine (Thr; T); (h) tryptophan (Trp; W); (i) valine (Val; V); (j) arginine (Arg; R); (k) cysteine (Cys; C); (l) glutamine (Gln; Q); (m) glycine (Gly; G); (n) proline (Pro; P); (o) serine (Ser; S); (p) tyrosine (Tyr; Y); (q) alanine (Ala; A); (r) asparagine (Asn; N); (s) aspartic acid (Asp; D); (t) glutamic acid (Glu; E); (u) selenocysteine (Sec; U); (v) pyrrolysine (Pyl; O). The term “non-proteinogenic amino acid” is used in the field of biochemistry to refer to naturally occurring and non-naturally occurring amino acids that are not proteinogenic amino acids, such as (1) citrulline (Cit); (2) cystine; (3) gamma-amino butyric acid (GABA); (4) ornithine (Orn); (5) theanine; (6) homocysteine (Hey); (7) thyroxine (Thx); and amino acid derivatives such as betaine; carnitine; carnosine creatine; hydroxytryptophan; hydroxyproline (Hyp); N-acetyl cysteine; S-Adenosyl methionine (SAM-e); taurine; tyramine, D-amino acids such as D-alanine (D-Ala); Norleucine (Nle); 4-hydroxyproline (HYP); 3,4-dehydro-L-proline (DHP); aminoheptanoic acid (AHP); (2R,5S)-5-phenyl-pyrrolidine-2-carboxylic acid (2PP); L-a-methylserine (MS); N-methylvaline (MV); 6-aminohexanoic acid (6-AHP); and 7-aminoheptanoic acid (7-AHP). Abbreviations for amino acid residues are used in keeping with standard polypeptide nomenclature delineated in IUPAC-IUB Biochem. Nom., J. Biol. Chem. 241: 527, 1966.

As used herein, “amino acid residue” means the individual amino acid units incorporated into a polypeptide. Amino acid residues are generally preferred to be in the “L” isomeric form. However, residues in the “D” isomeric form can be substituted for any L-amino acid residue, as long as the desired functional property (e.g., antibody binding) is retained by the polypeptide. It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

A “domain” as used herein, is a portion of a protein that has a tertiary structure. The domain may be connected to other domains in the complete protein by short flexible regions of polypeptide. Alternatively, the domain may represent a functional portion. For instance, an immunoglobulin molecule contains heavy and light chains, each chain containing a series of similar, although not identical, amino acid sequences. Each of these repeats corresponds to a discrete, compactly folded region of protein structure known as a protein domain. The light chain is made up of two such immunoglobulin domains, whereas the heavy chain of the IgG antibody contains four. Moreover, the amino-terminal sequences of both the heavy and light chains vary greatly between different antibodies and the remaining domains are constant between immunoglobulin chains of the same isotype. The amino-terminal variable domains (V) of the heavy and light chains (VH and VL, respectively) confer on it the ability to bind specific antigen, while the constant domains (C domains) of the heavy and light chains (CH and CL, respectively) make up the C region. The multiple heavy-chain C domains are numbered from the amino-terminal end to the carboxy terminus, for example CH1, CH2, CH3, and so on.

A “conservative” amino acid substitution, as used herein, generally refer to substitution of one amino acid residue with another amino acid residue from within a recognized group which typically changes the structure of the peptide by biological activity of the peptide is substantially retained. Conservatively substituted amino acids can be identified using a variety of well know methods, such as a blocks substitution matrix (BLOSUM), e.g., BLOSUM62 matrix. BLOSUM is a substitution matrix used for sequence alignment of proteins, wherein an alignment score is used to map out relationship between evolutionarily divergent protein sequences. They are based on local alignments. For instance, a BLOSUM62 substitution matrix can be found in the world-wide-web URL NCBI.NLM.NIH.GOV/class/fieldguide/BLOSUM62 .txt, which is incorporated by reference. Exemplary amino acid substitutions can be found in Table 1.

TABLE 1
Exemplary amino acid substitutions

| Amino | Exemplary Conservative |
|---|---|
| Acid | Substitutions |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

As used herein, “substantially identical” in reference to an amino sequence or nucleotide sequence means that a candidate sequence is at least 70% sequence identity to the reference sequence over a given comparison window (e.g., 250 amino acids). Thus, substantially similar sequences include those having, for example, at least 80% sequence identity, at least 85%, 90%, 91%, 92%, 93%, 94%, 95%, 96%, 97%, 98%, or 99%, or greater, e.g., 99.5%, sequence identity. Two sequences that are identical to each other are also substantially similar. The comparison window or the length of comparison sequence will generally be at least the length of antibody binding fragment of the candidate. Sequence identity is calculated based on the reference sequence, and algorithms for sequence analysis are known in the art. Thus, to determine percent sequence identity of two amino acid sequences, the sequences are aligned for optimal comparison purposes (e.g., gaps can be introduced in the sequence of one polypeptide for optimal alignment with the other polypeptide). The amino acid residues at corresponding amino acid positions are then compared. When a position in one sequence is occupied by the same amino acid residue as the corresponding position in the other sequence, then the molecules are identical at that position. The percent sequence identity between the two sequences is a function of the number of identical positions shared by the sequences (i.e., percent sequence identity=numbers of identical positions/total numbers of positions×100). Percent sequence identity between two polypeptide sequences can be determined using the Vector NTI software package (Invitrogen Corp., Carlsbad, Calif.). A gap opening penalty of 10 and a gap extension penalty of 0.1 are used for determining the percent identity of two polypeptides. All other parameters are set at the default.

As used herein, the term “derivative” includes salts, amides, esters, enol ethers, enol esters, acetals, ketals, acids, bases, solvates, hydrates, polymorphs or prodrugs of the individual amino acids, antigenic peptides or antibodies (or their antigen-binding fragments). Derivatives may be readily prepared by those of skill in this art using known methods for such derivatization. The derivatives suitable for use in the methods described herein may be administered to animals or humans without substantial toxic effects and either are biologically active or are prodrugs. Derivatives include solvent addition forms, e.g., a solvate or alcoholate. Derivatives further include amides or esters of the amino acids and/or isomers (e.g., tautomers or stereoisomers).

As used herein, “amino acid analogs” are compounds that are structurally or chemically similar to an amino acid. Many suitable amino acid analogs are known in the art, and representative examples include, e.g., p-Acetylphenylalanine, m-Acetylphenylalanine, O-allyltyrosine, Phenyl selenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-Iodophenylalanine, p-Bromophenylalanine, p-Nitrophenylalanine, L-DOPA, 3-Aminotyrosine, 3-Iodotyrosine, p-Isopropylphenylalanine, 3-(2-Naphthyl)alanine, biphenylalanine, homoglutamine, D-tyrosine, p-Hydroxyphenyllactic acid, 2-Aminocaprylic acid, bipyridylalanine, HQ-alanine, p-B enzoylphenylalanine, o-Nitrobenzylcysteine, o-Nitrobenzyl serine, 4,5-Dimethoxy-2-Nitrobenzyl serine, o-Nitrobenzyllysine, o-Nitrobenzyltyrosine, 2-Nitrophenylalanine, dansylalanine, p-Carboxymethylphenylalanine, 3-Nitrotyrosine, sulfotyrosine, acetyllysine, methylhistidine, 2-Aminononanoic acid, 2-Aminodecanoic acid, pyrrolysine, Cbz-lysine, Boc-lysine, allyloxycarbonyllysine, arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5,-triiodothyronine, and 3,3′,5,5′-tetraiodothyronine. The term includes modified or unusual amino acids e.g., D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, -phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid; functionalized amino acids, e.g., alkyne-functionalized, azide-functionalized, ketone-functionalized, aminooxy-functionalized amino acids and the like. See Liu et al., Ann. Rev. Biochem. 79:413, 2010; Kim et al., Curr. Opin. Chem. Biol., 17:412, 2013.

As used herein, the term “peptoid” refers to a polypeptide containing one or more N-substituted glycine residues. An N-substituted amino acid residue has a standard amino acid side-chain pendant from the N, rather than from the a-carbon. Representative examples of peptoids are provided in, e.g., U.S. Pat. Nos. 6,075,121 and 6,887,845.

As used herein, the term “peptidogylcan” refers to a rigid mesh made up of ropelike linear polysaccharide chains cross-linked by peptides.

The terms “polynucleotide” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other nonnucleotidic backbones, for example, polyamide (e.g., peptide nucleic acids (PNAs)) and polymorpholino polymers (commercially available from the Anti-Virals, Inc., Corvallis, OR, USA, as NEUGENE), and other synthetic sequence-specific nucleic acid polymers provided that the polymers contain nucleobases in a configuration which allows for base pairing and base stacking, such as is found in DNA and RNA. There is no intended distinction in length between the terms “polynucleotide” and “nucleic acid molecule.”

As used herein, the term “nucleotide” refers to molecules that, when joined, make up the individual structural units of the nucleic acids RNA and DNA. A nucleotide is composed of a nucleobase (nitrogenous base), a five-carbon sugar (either ribose or 2-deoxyribose), and one phosphate group. “Nucleic acids” as used herein are polymeric macromolecules made from nucleotide monomers. In DNA, the purine bases are adenine (A) and guanine (G), while the pyrimidines are thymine (T) and cytosine (C). RNA uses uracil (U) in place of thymine (T).

As used herein, a “nucleic acid,” “polynucleotide,” or “oligonucleotide” can be a polymeric form of nucleotides of any length, can be DNA or RNA, and can be single- or double-stranded. Nucleic acids can include promoters or other regulatory sequences. Oligonucleotides can be prepared by synthetic means. Nucleic acids include segments of DNA, or their complements spanning or flanking any one of the polymorphic sites. The segments can be between 5 and 1000 contiguous bases and can range from a lower limit of 5, 20, 50, 100, 200, 300, 500, 700 or 1000 nucleotides to an upper limit of 500, 1000, 2000, 5000, or 10000 nucleotides (where the upper limit is greater than the lower limit). Nucleic acids between 5-20, 50-100, 50-200, 100-200, 120-300, 150-300, 100-500, 200-500, or 200-1000 bases are common. A reference to the sequence of one strand of a double-stranded nucleic acid defines the complementary sequence and except where otherwise clear from context, a reference to one strand of a nucleic acid also refers to its complement. Complementation can occur in any manner, e.g., DNA=DNA; DNA=RNA; RNA=DNA; RNA=RNA, wherein, in each case, the “=” indicates complementation. Complementation can occur between two strands or a single strand of the same or different molecule.

As used herein, the term “hybridization” refers to any process by which a strand of nucleic acid bonds with a complementary strand through base pairing. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature. Variations on the above ranges and conditions are well known in the art.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay.

As used herein, the term “operably linked” refer to functionally related nucleic acid sequences. A promoter is operably linked with a coding sequence if the promoter controls the transcription of the encoded polypeptide. While operably linked nucleic acid sequences can be contiguous and in reading frame, certain elements, e.g., repressor genes, may not be contiguously linked but still bind to operator sequences that control expression of the polypeptide product.

As used herein, the term “vector” refers to a molecule that is capable of transferring nucleic acid sequences to target cells (e.g., viral vectors, non-viral vectors, particulate carriers, and liposomes). Typically, “vector construct” means any nucleic acid construct capable of directing the expression of a nucleic acid of interest and which can transfer nucleic acid sequences to target cells. Thus, the term includes cloning, expression, and viral vectors.

As used herein, the term “reporter” refers to molecule, e.g., a DNA, RNA, and/or polypeptide sequence, that is detectable in any detection system, including, but not limited to enzyme (e.g., ELISA, as well as other histochemical assays), fluorescent, and luminescent systems. Exemplary reporters include, e.g., β-glucuronidase, green fluorescent protein (GFP), E. coli β-galactosidase (LacZ), Halobacterium β-galactosidase, Neuropsora tyrosinase, human placental alkaline phosphatase, and chloramphenicol acetyl transferase (CAT), Aequorin (jellyfish bioluminescence), Firefly luciferase (EC 1.13.12.7) form Photinus pyralis, Renilla luciferase (EC 1.13.12.5) from the sea pansy Renilla reniformis, and Bacterial luciferase (EC 1.14.14.3) from Photobacterium fischeri. Preferably, the reporter comprises a luciferin-luciferase system. As used herein, the term “luciferin-luciferase system” refers to any process or method that allows the contact of luciferin and luciferase in the presence of a substrate (i.e., for example, cAMP) under conditions such that the resulting luminescence may be detected. Such a system may be comprised within a transfected host cell or provided in separate kit containers whereby the contents may be mixed together. “Reporters” includes the terms “Label” and “Detectable label” which when used herein refers to any moiety that, when attached to a moiety described herein, e.g., a peptide, protein or antibody, renders such a moiety detectable using known detection methods, e.g., spectroscopic, photochemical, electrochemiluminescent, and electrophoretic methods.

Various labels suitable for use in the present disclosure include labels which produce a signal through either chemical or physical means, wherein the signal is detectable by visual or instrumental means. Exemplary labels include, but are not limited to, fluorophores and radioisotopes. Such labels allow direct detection of labeled compounds by a suitable detector. e.g., a fluorometer. Such labels can include enzymes and substrates, chromogens, catalysts, fluorescent compounds, chemiluminescent compounds, and radioactive labels. Typically, a visually detectable label is used, thereby providing for instrumental (e.g. spectrophotometer) readout of the amount of the analyte in the sample. Labels include enzymes such as horseradish peroxidase, B-galactosidase, and alkaline phosphatase. Suitable substrates include 3,3′,5,5′-tetramethylbenzidine (TMB) and 1,2 dioxetane. The method of detection will depend upon the labeled used, and will be apparent to those of skill in the art. As noted, examples of suitable direct labels include radiolabels, fluorophores, chromophores, chelating agents, particles, chemiluminescent agents and the like.

For such embodiments, the label may be a direct label, i.e., a label that itself is detectable or produces a detectable signal, or it may be an indirect label, i.e., a label that is detectable or produces a detectable signal in the presence of another compound. “Labeled second antibody” refers to an antibody that is attached to a detectable label. The label allows the antibody to produce a detectable signal that is related to the presence of analyte in the fluid sample.

Suitable radiolabels include, by way of example and not limitation, ³H, ¹⁴C, ³²P, ³⁵S, ³⁶Cl, ¹³¹I and ¹⁸⁶Re.

As used herein, a “consensus” amino acid is an amino acid chosen to occupy a given position in the consensus polypeptide obtained by this method. A system which is organized to select consensus amino acids as described above may be a computer program, or a combination of one or more computer programs with “by hand” analysis and calculation. A set of amino acid sequences existing within the group of amino acid sequences from which the consensus sequence is prepared means a set of such sequences which are more similar to each other than to other members of the group, based on the evolutionary similarity analysis performed above. An example of such a group is a species where a set with in the group would be members of a particular polypeptide, e.g., antigenic regions.

As used herein, the term “fusion protein” refers to a peptide or a functional fragment thereof, that is bonded through a bond, e.g., a peptide bond (or amide bond), to an amino acid sequence that is not bonded naturally in the parent peptide. Illustrative fusion polypeptides include fusions of the antibodies of the disclosure (or antigen-binding fragment thereof) with an enzyme (e.g., alkaline phosphatase; AP).

As pertains to the present disclosure, a biological fluid can be a solid, or semi-solid sample, including feces, biopsy specimens, skin, nails, and hair, or a liquid sample, such as urine, saliva, sputum, mucous, blood, blood components such as plasma or serum, amniotic fluid, semen, vaginal secretions, tears, spinal fluid, washings, and other bodily fluids. Included among the sample are swab specimens from, e.g., the cervix, urethra, nostril, and throat. Any of such samples may be from a living, dead, or dying animal or a plant. Animals include mammals, such as humans.

“Urine” refers to liquid excrement voided through the urethra or collected from a catheter from a patient.

“Antibody” refers to a polypeptide substantially encoded by an immunoglobulin gene or immunoglobulin genes, or fragments thereof. The recognized immunoglobulin genes include the kappa, lambda, alpha, gamma, delta, epsilon, and mu constant regions, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, an antibody is an immunoglobulin having an area on its surface or in a cavity that specifically binds to and is thereby defined as complementary with a particular spatial and polar organization of another molecule. The antibody can be polyclonal or monoclonal (abbreviated as mAb or moAb). Antibodies may include a complete immunoglobulin or fragments thereof. Fragments thereof may include Fab, Fv and F(ab′)2. Fab′, and the like. Antibodies may also include chimeric antibodies or fragment thereof made by recombinant methods. “Antibody” includes whole antibodies, including those of the IgG, IgM and IgA isotypes, and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The IgG heavy chain constant region is comprised of four domains. CH1, hinge, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR).

As used herein, the term “complementarity determining region” or “CDR” refers to the hypervariable region amino acid residues of an antibody which are responsible for antigen-binding. The hypervariable region or CDRs of the human IgG subtype of antibody typically comprise amino acid residues from residues 24-34 (L1), 50-56 (L2) and 89-97 (L3) in the light chain variable domain and 31-35 (H1), 50-65 (H2) and 95-102 (H3) in the heavy chain variable domain as described by Kabat et al. (supra) and/or those residues from a hypervariable loop in the heavy chain variable domain as described by Chothia et al. (J. Mol. Biol. 196: 901-17, 1987). Framework or FR residues are those variable domain residues other than and bracketing the hypervariable regions.

Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, the term “single domain antibody” or “sdAb” refers to a type of single chain antibody comprising a variable region (VHH) of a heavy chain of a human antibody. SdAbs are antibody fragments consisting of a single monomeric variable antibody domain. They are derived, for example, from heavy chain antibodies derived from humans, which consist only of two antibody heavy chains, with no light chain. With a molecular weight of only 12-15 kDa, sdAbs are much smaller than monoclonal antibodies (mAbs), e.g., IgG antibodies (150-160 kDa), which have two heavy protein chains and two light chains. SdAbs may be derived from any species including, but not limited to mouse, human, camel, llama, goat, rabbit, bovine. The sdAb can be modified versions of a naturally occurring immunoglobulin known as heavy chain antibody devoid of light chains. Such immunoglobulins are disclosed in U.S. Pat. Nos. 8,293,233 and 9,371,371; and U.S. Pub. No. 2011-0052565. For clarity reasons, the variable domain derived from a heavy chain antibody naturally devoid of light chain is known herein as a V_{HH} or sdAb to distinguish it from the conventional V_{H} of four chain immunoglobulins.

As used herein, the term “epitope” means a protein determinant capable of specific binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The term “native conformational epitope” or “native protein epitope” are used interchangeably herein, and include protein epitopes resulting from conformational folding of the antigen which arise when amino acids from differing portions of the linear sequence of the antigen come together in close proximity in 3-dimensional space. Such conformational epitopes are distributed on the extracellular side of the plasma membrane.

“Isolated antibody,” as used herein, is intended to refer to an antibody which is substantially free of other antibodies having different antigenic specificities. An isolated antibody that specifically binds to an epitope, isoform or variant may, however, have cross-reactivity to other related antigens. Moreover, an isolated antibody may be substantially free of other cellular material and/or chemicals. In some embodiments, a combination of “isolated” monoclonal antibodies having different specificities are combined in a well-defined composition.

“Immunological binding,” as used herein, generally refers to the non-covalent interactions of the type that occurs between an antibody, or fragment thereof, and the type 1 interferon or receptor for which the antibody is specific. The strength, or affinity, of immunological binding interactions can be expressed in terms of the dissociation constant (K_{D}) of the interaction, wherein a smaller K_{D} represents a greater affinity. Immunological binding properties of selected antibodies can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant Kd. See, generally, Davies et al., Annual Rev. Biochem. 59:439-473 (1990).

“Specific binding” or “specifically binds” refers to antibody binding to a predetermined antigen. Typically, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”.

“High affinity” for an IgG antibody refers to an antibody having a K_{D} of 10⁻⁸ M or less, more preferably 10⁻⁹ M or less and even more preferably 10⁻¹⁰ M or less. However, “high affinity” binding can vary for other antibody isotypes. For example, “high affinity” binding for an IgM isotype refers to an antibody having a K_{D} of 10⁻⁷ M or less, more preferably 10⁻⁸ M or less.

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity that have variable and constant regions derived from human germline immunoglobulin sequences. Monoclonal antibodies to a compound may be prepared using any technique which provides for the production of antibody molecules by continuous cell lines in culture. These include, but are not limited to, the hybridoma technique originally described by Kohler & Milstein, 1975, Nature 256:495-497 and/or Kaprowski, U.S. Pat. No. 4,376,110; the human B-cell hybridoma technique described by Kosbor et al., 1983, Immunology Today 4:72 and/or Cote et al., 1983, Proc. Natl. Acad. Sci. USA 80:2026-2030; and the EBV-hybridoma technique described by Cole et al., 1985, Monoclonal Antibodies and Cancer Therapy, Alan R. Liss, Inc., pp. 77-96. Alternatively, techniques described for the production of single chain antibodies (see, e.g., U.S. Pat. No. 4,946,778) can be adapted to produce compound-specific single chain antibodies.

As used herein, the term “hybridoma” refers to cells produced by fusing two cell types together. Commonly used hybridomas include those created by the fusion of antibody-secreting B cells from an immunized animal, with a malignant myeloma cell line capable of indefinite growth in vitro. These cells are cloned and used to prepare monoclonal antibodies.

Antibody fragments which contain deletions of specific binding sites may be generated by known techniques. For example, such fragments include but are not limited to F(ab′)2 fragments, which can be produced by pepsin digestion of the antibody molecule and Fab fragments, which can be generated by reducing the disulfide bridges of the F(ab′)2 fragments. Alternatively, Fab expression libraries may be constructed (Huse et al., 1989, Science 246:1275-1281) to allow rapid and easy identification of monoclonal Fab fragments with the desired specificity for the peptide of interest.

As used herein, the term “recombinant antibody” refers to antibodies that are prepared, expressed, created or isolated by recombinant means, such as (a) antibodies isolated from an animal or a hybridoma prepared therefrom (described further below), (b) antibodies isolated from a host cell transformed to express the antibody, e.g., from a transfectoma, (c) antibodies isolated from a recombinant, combinatorial human or other species antibody library, and (d) antibodies prepared, expressed, created or isolated by any other means that involve splicing of immunoglobulin gene sequences to other DNA sequences.

The antibody or antibody fragment specific for the desired peptide can be attached, for example, to agarose, and the antibody-agarose complex is used in immunochromatography to purify peptides. See, Scopes, 1984, Protein Purification: Principles and Practice, Springer-Verlag New York, Inc., N.Y., Livingstone, 1974, Methods In Enzymology: Immunoaffinity Chromatography of Proteins 34:723-731.

As used herein, the term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the disclosure includes bispecific, trispecific, tetraspecific, and other multispecific molecules which are directed to cell surface antigens, such as, e.g., GAC, and to other targets, such as M proteins.

As used herein, the term “bivalent” antibody refers to antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al., PNAS USA, 90, 6444-8, 1993; Poljak et al., Structure, 2, 1121-23, 1994). “Multivalent” antibodies include two or more binding domains which may all be of the same specificity or may have multiple specificities.

As used herein, “chimeric antibodies” are those antibodies that retain distinct domains, usually the variable domain, from one species and the remainder from another species; e.g., mouse-human chimeras. The term “human antibody”, as used herein, is intended to include antibodies having variable and constant regions derived from or closely matching human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo such as during the recombination of V, D, and J segments of the human heavy chain). Thus as used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially similar to those encoded by human germline antibody genes. Human antibodies have been classified into groupings based on their amino acid sequence similarities (Nikoloudis et al., Peer J., 2, e456, 2014; Adolf-Bryfogle et al., Nucleic Acids Res., 43, D432-8, 2015). Thus, using a sequence similarity search, an antibody with similar linear sequence can be chosen as a template to select or create human or humanized antibodies. Techniques for the production of chimeric antibodies are further described in Morrison et al., 1984, Proc. Natl. Acad. Sci. USA 81:6851-6855; Neuberger et al., 1984, Nature 312:604-608; Takeda et al., 1985, Nature 314:452-454; Boss, U.S. Pat. No. 4,816,397; Cabilly, U.S. Pat. No. 4,816,567).

As used herein, “humanization” for making humanized antibodies (also called reshaping or CDR-grafting) includes established techniques for reducing the immunogenicity of monoclonal antibodies (mAbs) from xenogeneic sources (commonly rodent) and for improving affinity or the effector functions (ADCC, complement activation, C1q binding). The engineered MAb can be produced using the techniques of molecular biology, using phage displayed randomized sequences, or synthesized de novo. For example, in order to humanize an antibody with incorporated the CDR regions from a nonhuman species, the design might include variations such as conservative amino acid substitutions in residues of the CDRs, and back substitution of residues from the nonhuman MAb into the human framework regions (back-mutations). The positions can be discerned or identified by sequence comparison methods, consensus sequence analysis, or structural analysis of the variable regions' 3D structure. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR (framework) residues can be selected and combined from the consensus and import sequences so that the desired antibody characteristic, such as increased affinity for the target antigen(s), is achieved. As the datasets of known parameters for antibody structures increases, so does the sophistication and refinement of these techniques. Another approach to humanization is to modify only surface residues of the rodent sequence with the most common residues found in human mAbs and has been termed “resurfacing” or “veneering.” Known human Ig sequences are disclosed in, e.g., IGBLAST (NCBI); Kabat et al., Sequences of Proteins of Immunological Interest, DIANE Publishing, 1992. Humanization or engineering of antibodies of the present disclosure can be performed using any known method, such as but not limited to those described in, Jones et al., Nature 321:522 (1986); Riechmann et al., Nature 332:323 (1988); Verhoeyen et al., Science 239:1534 (1988)), Sims et al., J. Immunol. 151: 2296 (1993); Chothia et al., J. Mol. Biol., 196:901 (1987), Carter et al., PNAS USA, 89:4285 (1992); Presta et al., J. Immunol., 151:2623 (1993), U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; WO199900683; and WO1994018219.

As used herein, the term “pharmaceutically acceptable” means a molecule or a material that is not biologically or otherwise undesirable, i.e., the molecule or the material can be administered to a subject without causing any undesirable biological effects such as toxicity.

As used herein, the term “carrier” denotes buffers, adjuvants, dispersing agents, diluents, and the like. For instance, the peptides or compounds of the disclosure can be formulated for administration in a pharmaceutical carrier in accordance with known techniques. See, e.g., Remington, The Science & Practice of Pharmacy (9^{th} Ed., 1995). In the manufacture of a pharmaceutical formulation according to the disclosure, the peptide or the compound (including the physiologically acceptable salts thereof) is typically admixed with, inter alia, an acceptable carrier. The carrier can be a solid or a liquid, or both, and is preferably formulated with the peptide or the compound as a unit-dose formulation, for example, a tablet, which can contain from about 0.01 or 0.5% to about 95% or 99%, particularly from about 1% to about 50%, and especially from about 2% to about 20% by weight of the peptide or the compound. One or more peptides or compounds can be incorporated in the formulations of the disclosure, which can be prepared by any of the well-known techniques of pharmacy.

As used herein, the term “culture,” refers to any sample or specimen which is suspected of containing one or more microorganisms or cells. “Pure cultures” are cultures in which the cells or organisms are only of a particular species or genus. This is in contrast to “mixed cultures,” wherein more than one genus or species of microorganism or cell are present.

“Detect” and “detection” have their standard meaning, and are intended to encompass detection, measurement and/or characterization of a selected protein or protein activity. For example, enzyme activity may be “detected” in the course of detecting, screening for, or characterizing inhibitors, activators, and modulators of the protein.

The term “reference level” refers to a reference level that can be previously obtained from the subject, from another subject, or can refer to a numerical value derived from multiple normal subjects not infected with the pathogen of interest. Appropriate reference levels can be measured and chosen according to techniques known to those skilled in the art.

As used herein, the terms “treat,” “treating,” or “treatment of,” refers to reduction of severity of a condition or at least partially improvement or modification thereof, e.g., via complete or partial alleviation, mitigation or decrease in at least one clinical symptom of the disease, disorder, or condition.

As used herein, the term “administering” is used in the broadest sense as giving or providing to a subject in need of the treatment, a composition such as the compound or peptide of the disclosure, or a pharmaceutical composition containing the peptide or the compound. For instance, in the pharmaceutical sense, “administering” means applying as a remedy, such as by the placement of a peptide or an antibody in a manner in which such molecule would be received, e.g., intravenous, oral, topical, buccal (e.g., sub-lingual), vaginal, parenteral (e.g., subcutaneous; intramuscular including skeletal muscle, cardiac muscle, diaphragm muscle and smooth muscle; intradermal; intravenous; or intraperitoneal), topical (i.e., both skin and mucosal surfaces), intranasal, transdermal, intraarticular, intrathecal, inhalation, intraportal delivery, organ injection (e.g., eye or blood, etc.), or ex vivo (e.g., via immunoapheresis).

As used herein, “contacting” means that the composition comprising the active ingredient is introduced into a sample containing a target, e.g., cell target, in a test tube, flask, tissue culture, chip, array, plate, microplate, capillary, or the like, and incubated at a temperature and time sufficient to permit binding of the antigen (e.g., GAC) or a test compound (e.g., NAG) to the target (e.g., antibodies) or vice versa. In the in vivo context, “contacting” means that the diagnostic or therapeutic molecule is introduced into a patient or a subject for the diagnosis or treatment of a disease, and the molecule is allowed to come in contact with the patient's target tissue, e.g., blood tissue, in vivo or ex vivo.

As used herein, the term “therapeutically effective amount” refers to an amount that provides some improvement or benefit to the subject. Alternatively stated, a “therapeutically effective” amount is an amount that will provide some alleviation, mitigation, or decrease in at least one clinical symptom in the subject. Methods for determining therapeutically effective amount of the therapeutic molecules, e.g., antibodies, are described below.

As used herein, the term “inhibit” refers to reduction in the amount, levels, density, turnover, association, dissociation, activity, signaling, or any other feature associated with an etiological agent of a disorder.

As used herein, the term “subject” means an individual. In one aspect, a subject is a mammal such as a human. In one aspect a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., llama, horses, cows), wild animals (e.g., deer, elk, moose, etc.,), laboratory animals (e.g., mouse, rabbit, rat, gerbil, guinea pig, etc.) and avian species (e.g., chickens, turkeys, ducks, etc.). Subjects can also include, but are not limited to fish, amphibians and reptiles. Subjects may further include invertebrates such as ticks, lice, and fleas. Preferably, the subject is a human subject. More preferably, the subject is a human patient.

As used herein, the term “detecting,” refers to the process of determining a value or set of values associated with a sample by measurement of one or more parameters in a sample, and may further comprise comparing a test sample against reference sample. In accordance with the present disclosure, the detection of a disease or disorder in a subject may include identification, assaying, measuring and/or quantifying one or more antigens in the subject's biological sample, e.g., urine, saliva, sputum, phlegm, nasal discharge, mucus, tears, blood, or serum.

As used herein, the term “diagnosis” refers to methods by which a determination can be made as to whether a subject is likely to be suffering from a given disease or condition, including but not limited diseases or conditions characterized by antigens or pathogens. The skilled artisan often makes a diagnosis on the basis of one or more diagnostic indicators, the presence, absence, amount, or change in amount of which is indicative of the presence, severity, or absence of the disease or condition. Other diagnostic indicators can include patient history; physical symptoms and the like. Diagnostic methods of the disclosure can be used independently, or in combination with other diagnosing methods, to determine whether a course or outcome is more likely to occur in a patient exhibiting a given characteristic.

As used herein, the term “cell” is used interchangeably with the term “biological cell.” Non-limiting examples of biological cells include eukaryotic cells, plant cells, animal cells, such as mammalian cells, insect cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immune cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, e.g., from a human, a mouse, a rat, a horse, a goat, a sheep, a cow, a primate, etc.

As used herein, the term “sample” refers to a composition that is obtained or derived from a subject of interest that contains a cellular and/or other molecular entity that is to be characterized and/or identified, for example based on physical, biochemical, chemical and/or physiological characteristics.

As used herein a “biological sample” is a substance obtained from the subject's body. The particular “biological sample” selected will vary based on the disorder the patient is suspected of having and, accordingly, which biological sample is most likely to contain the analyte. The source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Preferably, the sample is obtained from pulmonary organs, including, e.g., saliva, sputum, phlegm, nasal discharge, mucus, pleural fluid, broncho-alveolar lavage, blood, etc.

The term “susceptible” or “predisposition” as used herein describes a subject at risk for developing an infection, disease or disorder. These terms can be used to mean that a subject having a particular genotype and/or haplotype has a higher likelihood than one not having such a genotype and/or haplotype for developing a particular disease or disorder.

“Ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

“Chromophore” refers to a moiety with absorption characteristics, i.e., are capable of excitation upon irradiation by any of a variety of photonic sources. Chromophores can be fluorescing or nonfluorescing, and includes, among others, dyes, fluorophores, luminescent, chemiluminescent, and electrochemiluminescent molecules.

Examples of suitable indirect labels include enzymes capable of reacting with or interacting with a substrate to produce a detectable signal (such as those used in ELISA and EMIT immunoassays), ligands capable of binding a labeled moiety, and the like. Suitable enzymes useful as indirect labels include, by way of example and not limitation, alkaline phosphatase, horseradish peroxidase, lysozyme, glucose-6-phosphate dehydrogenase, lactate dehydrogenase and urease. The use of these enzymes in ELISA and EMIT immunoassays is described in detail in Engvall, 1980, Methods Enzym. 70: 419-439 and U.S. Pat. No. 4,857,453.

“Substrate,” “Support,” “Solid Support,” “Solid Carrier,” or “Resin” are interchangeable terms and refer to any solid phase material. Substrate also encompasses terms such as “solid phase,” “surface,” and/or “membrane.” A solid support can be composed of organic polymers such as polystyrene, polyethylene, polypropylene, polyfluoroethylene, polyethleneoxy, and polyacrylamide, as well as co-polymers and grafts thereof. A solid support can also be inorganic, such as glass, silica, controlled pore glass (CPG), reverse phase silica or metal, such as gold or platinum. “Solid support” includes membranes (e.g. nitrocellulose), microtiter plate (e.g. PVC, polypropylene, polystyrene), dipstick, test tube, and glass or plastic beads. The configuration of a substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or, other container, vessel, feature, or location. A plurality of supports can be configured on an array at various locations, addressable for robotic delivery of reagents, or by detection methods and/or instruments. Methods for immobilizing biomolecules are well known in the art, and the antibody can be attached covalently, or non-covalently. In one embodiment, the solid support is a streptavidin coated plate to which a biotinylated antibody is non-covalently attached.

In statistics and diagnostic testing, sensitivity and specificity are statistical measures of the performance of a binary classification test. Sensitivity (also called “recall rate”) measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition). These two measures are closely related to the concepts of type I and type II errors. A theoretical, optimal prediction aims to achieve 100% sensitivity (i.e. predict all people from the sick group as sick) and 100% specificity (i.e. not predict anyone from the healthy group as sick), however theoretically any predictor will possess a minimum error bound known as the Bayes error rate.

“Specificity” relates to the ability of the diagnostic test to identify negative results.

$$Specificity = \frac{\# of True Negatives}{\# of True Negatives + \# of False Positives}$$

If a test has high specificity, a positive result from the test cans a high probability of the presence of the disease for which the test is testing.

“Sensitivity” relates to the ability of the diagnostic test to identify positive results.

$$Sensitivity = \frac{\# of True Positives}{\# of True Positives + \# of False Negatives}$$

If a test has high sensitivity, a negative result would suggest the absence of disease. For example, a sensitivity of 100% means that the test recognizes all actual positives—i.e. all sick people are recognized as being ill. Thus, in contrast to a high specificity test, negative results in a high sensitivity test are used to rule out the disease.

For any test, there is usually a trade-off between the measures. For example: in an airport security setting in which one is testing for potential threats to safety, scanners may be set to trigger on low-risk items like belt buckles and keys (low specificity), in order to reduce the risk of missing objects that do pose a threat to the aircraft and those aboard (high sensitivity). This trade-off can be represented graphically using a receiver operating characteristic (ROC) curve.

In some embodiments, a ROC is used to generate a summary statistic. Some common versions are: the intercept of the ROC curve with the line at 90 degrees to the no-discrimination line (also called Youden's J statistic); the area between the ROC curve and the no-discrimination line; the area under the ROC curve, or “AUC” (“Area Under Curve”), or A′ (pronounced “a-prime”); d′ (pronounced “d-prime”), the distance between the mean of the distribution of activity in the system under noise-alone conditions and its distribution under signal-alone conditions, divided by their standard deviation, under the assumption that both these distributions are normal with the same standard deviation. Under these assumptions, it can be proved that the shape of the ROC depends only on d′.

The “positive predictive value (PPV),” or “precision rate” of a test is a summary statistic used to describe the proportion of subjects with positive test results who are correctly diagnosed. It is a measure of the performance of a diagnostic method, as it reflects the probability that a positive test reflects the underlying condition being tested for. Its value does however depend on the prevalence of the outcome of interest, which may be unknown for a particular target population.

The PPV can be derived using Bayes' theorem. The PPV is defined as:

PPV=# of True Positives=# of True Positives

# of True Positives+# of False Positives# of Positive calls

where a “true positive” is the event that the test makes a positive prediction, and the subject has a positive result under the gold standard, and a “false positive” is the event that the test makes a positive prediction, and the subject has a positive result under the gold standard.

“Negative predictive value (NPV)” is defined as the proportion of subjects with a negative test result who are correctly diagnosed. A high NPV means that when the test yields a negative result, it is uncommon that the result should have been positive. In the familiar context of medical testing, a high NPV means that the test only rarely misclassifies a sick person as being healthy. Note that this says nothing about the tendency of the test to mistakenly classify a healthy person as being sick.

The NPV is also defined as:

$$NPV = \# of True Negatives = \# of True Negatives \# of True Negatives + \# of False Negatives \# of Negative calls$$

where a “true negative” is the event that the test makes a negative prediction, and the subject has a negative result under the gold standard, and a “false negative” is the event that the test makes a negative prediction, and the subject has a positive result under the gold standard. If the prevalence, sensitivity, and specificity are known, the positive and negative predictive values (PPV and NPV) can be calculated for any prevalence as follows:

$$PPV = \frac{sensitivity \times prevalence}{\begin{matrix}sensitivity \times prevalence + ( 1 - specificity ) \times \\ ( 1 - prevalence )\end{matrix}}$$

If the prevalence of the disease is very low, the positive predictive value will not be close to 1 even if both the sensitivity and specificity are high. Thus in screening the general population it is inevitable that many people with positive test results will be false positives.

The rarer the abnormality, the more sure one can be that a negative test indicates no abnormality, and the less sure that a positive result really indicates an abnormality. The prevalence can be interpreted as the probability before the test is carried out that the subject has the disease, known as the prior probability of disease. The positive and negative predictive values are the revised estimates of the same probability for those subjects who are positive and negative on the test, and are known as posterior probabilities. The difference between the prior and posterior probabilities is one way of assessing the usefulness of the test.

For any test result we can compare the probability of getting that result if the patient truly had the condition of interest with the corresponding probability if he or she were healthy. The ratio of these probabilities is called the likelihood ratio, calculated as sensitivity/(1-specificity). (Altman D G, Bland J M (1994). “Diagnostic tests 2: Predictive values”. BMJ 309 (6947): 102).

“Rule-out criteria” “Rule-Out,” or “RO” are terms used in a medical differential diagnosis of a disease or condition, in which certain criteria are evaluated in a clinical decision-making process of elimination or inclusion. A subject is “ruled-out” when, upon consideration of the criteria, the subject has been determined not to have met all or a significant number of criteria for having a disease.

Methods of Detection and/or Diagnosis

Accordingly, in one aspect of the disclosure, a method for diagnosing and/or detecting a disease or disorder in a human subject is provided. The method comprises providing a immunoassay comprising a fusion antibody or binding agent as described herein, and contacting the immunoassay with a sample from a subject.

In another aspect, a method is provided for ruling out a disease or disorder in a human subject, in which method a sample of a body fluid is obtained from the subject; the sample is contacted with a fusion antibody according to the invention to determine whether the antibody fusion detects the presence of an antigen or target in the sample; and, for those human subjects for which no presence is detected, the disease or disorder is ruled out.

In some embodiments, the body fluid sampled is urine. In some embodiments, the body fluid sampled is urine. In some embodiments, the body fluid sampled is blood. In some embodiments, the body fluid sampled is sputum.

### Kits

Kits for detecting substances present in solid, semi-solid, or liquid biological samples are also provided. The kits may include instructions for obtaining biological samples and contacting them with sample buffer, for mixing the samples with sample buffer, placing labels on the apparatus and recording relevant test data; for shipping the apparatus, and the like. The kits may include instructions for reading and interpreting the results of an assay. The kits may further comprise reference samples that may be used to compare test results with the specimen samples.

It will be appreciated that this antibody can potentially be engineered with genetically fused tags such as NanoGlo®, NanoLuc° , SEAP and GFP for high sensitivity assay detection. Luminescent or fluorescent tag detection technology offers maximal sensitivity, high intensity signal, low background, wide dynamic range, rapid signal production, and assay format compatibility for next-gen immunoassay development.

The current antibody fusions are suitable for use in the systems sold under the brand name Nano-Glo® Luciferase Assay System. This system provides a simple, single-addition reagent that generates a glow-type signal in the presence of NanoLuc® luciferase; half-life is approximately 120 minutes in commonly used tissue culture media. The reagent is prepared by mixing Nano-Gb® Luciferase Assay Substrate and NanoGlo® Luciferase Assay Buffer. The reagent contains an integral lysis buffer allowing use directly on cells expressing NanoLuc® luciferase or the culture media when luciferase is secreted. NanoGlo® Luciferase Assay Reagent is a dedicated product for the detection of NanoLuc® Luciferase.

In another embodiment, the antibodies form part of a kit and/or an immunoassay for use in diagnosis. In one embodiment, the immunoassay provides a detectable signal that can be read visually or optically by an instrument. The detectable signal, in one embodiment, is a fluorescent signal, such a provided by a detection particle, such as a europium particle, attached to the antibody.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

### High Sensitivity Bioluminescence Immunoassay Detection System

Nanoluc® luciferase and SEAP (Secreted Embryonic Alkaline Phosphatase) offer very bright labels with a broad linear detection range detection (up to 1.0E10 RLU) for antibody-antigen detection systems. Each are well suited for bioluminescence detection, superior to colorimetric or fluorescent assays, while providing exceptional stability with improved sensitivity. PCT Fabs were engineered with genetically fused tags for high sensitivity assay detection development. Examples of suitable assay systems include:
(A) PCT antibody-based immunoassays using recombinantly expressed PCT MAb with a fused Nanoluc® luciferase. Nanoluc is a small highly active engineered luciferase protein, capable of an extremely high turnover rate with appropriate substrates. The engineered PCT Fab_Nluc fusion demonstrated broad linear range (up to 1.0E10 RLU), with stable signals. No significant signal decrease was observed after substrate incubation for 60 min. At 0.2 pg/ml Fab_Nluc concentration, a signal/noise ratio of >50 was observed
(B) PCT antibody-based immunoassays with engineered SEAP. SEAP (Secreted Embryonic Alkaline Phosphatase), a 50 kDa fusion tag enzyme, can be detected with a verity of substrates commercially available. The engineered PCT Fab_SEAP demonstrated broad linear range enzymatic activity (up to 1.0E10 RLU), with stable signals—no significant signal decrease was observed after substrate incubation for 30 min. At 0.1 pg/ml Fab_SEAP concentration, a signal/noise ratio >65 was observed.
(C) Thyroid TRAb-based immunoassays with a recombinantly expressed M22_Nluc fusion protein. Early studies with a beads-based and ELISA formats demonstrated a broad linear range of luminescence activity (up to 1.0E9 RLU), with stable signals—no significant signal decrease was observed after substrate incubation for 30 min. At 5 pg/ml the M22_Nluc reagent showed a signal/noise ratio >30 was observed.
(D) Lyme VlsE/C6, OspC/10 and DbpA antibody-based assays. Where each MAb is recombinantly expressed with a fused tag.

Fluorescence Resonance Energy Transfer (FRET) technology and genetically encoded FRET biosensor proteins. FRET technology and genetically encoded FRET biosensor proteins provide a powerful tool for commercial immunoassay development. Fluorescent proteins (e.g. GFPs or RFPs) are most commonly used as donor/acceptor fluorophores in FRET biosensors, particularly since FPs are genetically encodable and compatible. Methods to measure FRET pairs are well developed for immunoassays

M22 (TSHR-specific) antibody was recombinantly expressed with a fluorescent protein i.e., Green Fluorescent Protein (GFP) to the end of Fab heavy chain. The M22_Fab_GFP worked well on a ELISA-based assay system for detection. This is useful in lateral flow or assays with a wash step such as an ELISA and Flow-thru assays. It can also be applied to Flow Cytometry assays without the need for conjugation.

It will be appreciated that antibodies can be recombinantly expressed with a fluorescent protein such as GFP or RFP or labeled with RPE (R-phycoerythrin).

On a separate recombinant antigen or antibody (part of the same assay) a separate fluorescent protein or fluorophore can be expressed through a fusion tag that has an excitation wavelength that matches with the emission wavelength of the fluorescent protein of the first antigen/antibody. The resulting assay system would then support a homogeneous assay system (i.e., BRET or FRET-based). For example, the TSHR-specific monoclonal antibody M22 was fused to a sensitive label/detector and recombinantly produced as fusion proteins for immunoassay development.

Preliminary studies were performed with homogeneous formats. For example, M22_Nluc emitted luminescence at 460 nm (in the presence of substrate), and RPE-anti human lgG exhibited excitation at 480nm and emission at 575nm. When those two are mixed to allow binding to occur, close proximity resulted in fluorescence energy transfer that can be measured by reading at 575 nm as emission.

Enzyme Fragment Complementation assays, such as NanoBIT® from Promega can be adapted to the invention as well. Here, antibody and antigen serve as “bait” and “prey”—each are recombinantly or covalently linked to fragments of a third protein (e.g., Luciferase) which acts as a “reporter”. The interaction between the bait and the prey proteins brings the fragments of the reporter protein in close proximity to allow them to form a functional reporter protein whose enzymatic activity can then be measured. Luciferase-derived NanoBIT® (Large BIT® is a 156aa protein, while Small BIT® is an 11aa peptide) can be easily utilized in accordance with the invention. First, a recombinant antibody is expressed with a non-functional luminescent protein portion fused to the antibody heavy chain or the end of a Fab heavy chain or light chain. Next, a second fragment of the non-functional luminescent protein is linked to a second antigen or antibody such that the second fragment of the luminescent protein is capable of binding to the first luminescent protein portion. This results in a complementary functional protein. The binding occurs when, for example, the first antibody binds to an antigen that the second antibody binds to bringing the two parts of the luminescent protein in close proximity to one another, thus allowing binding to occur. This results in an active enzyme that reacts with substrate leading to signal generation.

### Example 2

### Bioluminescence Analyte Detection Systems for Thyroid Immunoassays

Detection methods were performed for providing enhanced sensitivity with improved precision for detecting antigen/antibody binding events. The effort was divided into two parts: first to explore and investigate antigen/antibody expression systems, and then to identify analyte detection systems for evaluation. This evaluation identified different luciferase based detection systems primarily developed and manufactured by Promega® Corp., for example, the firefly luciferase (Bright-Glo®). The GloSensor® by Promega was developed and commercialized based on the fusion & circular permutation of firefly luciferase, an improved intracellular cyclic AMP (cAMP) homogeneous detection system. Additionally suitable is the NanoLuc® luciferase (Nluc) system by Promega. See England et al., NanoLuc: a small luciferase is brightening up the field of bioluminescence. Bioconjug. Chem. 27, 1175-1187 (2016); and Boute et al., NanoLuc® luciferase—a multifunctional tool for high throughput antibody screening. See Front Pharmacol. 2016;7:27. This small (19 kDa), highly stable, ATP independent, bioluminescent protein became a robust ultra-high sensitivity detection system for assay development. This system is versatile, and allows for cellular, solid phase ELISA, and homogeneous assays with bioengineering of the enzyme and/or the use of BRET-based screening assays. The Nluc® protein luciferase provides improved Point-of-Care (POC) testing.

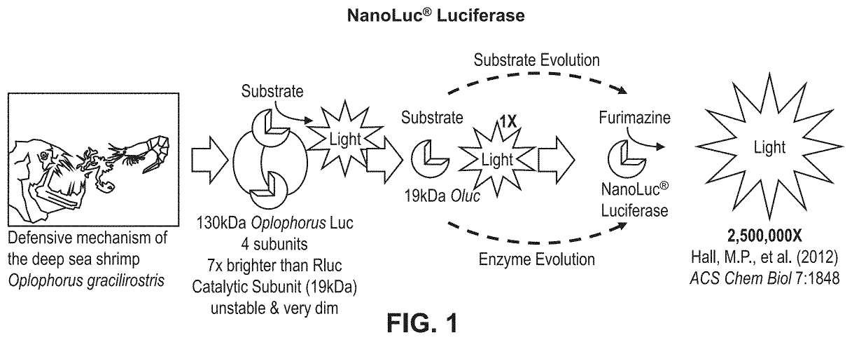

Beneficial properties of the NanoLuc luciferase properties include small size (19 kDa), thermal stability, activity over a broad pH range, monomeric structure, no PTM detection in mammalian cells, no formation of disulfide bonds, uniform distribution in cells, high brightness and broad Linear Dynamic Range. Additionally, NanoLuc is an ATP independent Glow-type Signal that provides a stable signal with no ramp-on rate, with a half-life >2 hr. FIG. 1 shows a summary of the Nanoluc® luciferase system.

### Example 3

A fusion protein of a synthetic fragment of TSHR that comprises amino acids 20-275 of the extracellular domain (ECD) of the TSHR is referred to herein as L1-10. The L1-10 fusion protein specifically binds to thyroid-stimulating antibody (M22, disclosed in U.S. Pat. No. 8,110,664; sequence incorporated by reference herein) and to a thyroid-blocking antibody (K1-70, disclosed in U.S. Pat. No. 9,073,992, sequence incorporated by reference herein) in BIACORE screening assays (data not shown). Following identification of desired antigens, and development of the L1-10 (TSH Receptor, THSR) and the anti-TSHR antibody (TRAb) reagents, an ELISA-based thyroid immunoassay was developed. To explore the bioluminescence technology, initial exploration studies were performed using the L1-10/TRAb M22 assay system. Two recombinant fusion constructs, M22_NLuc antibody and L1-10_NLuc antigen respectively, were designed and engineered with NLuc tag for mammalian expression. Subsequent protein sequences are listed below for construction. Proteins were affinity purified by Ni-NTA and StrepTactin technology respectively (at ATUM) and delivered for enzymatic and functional testing. Protein characterization was performed by SDS-PAGE and SEC-HPLC.

The L1-10 fusion protein has the following structure:

ψ-β-γ₁-ε-γ₂-π-PEP-ϕ (Formula I)

wherein,

ψ is a signal peptide or absent;

β is a binding molecule or absent;

γ₁ and γ₂ are each, independently of one another, linkers or absent;

ε is an expression enhancer or absent;

π is a cleavable site which is present or absent;

PEP is a polypeptide comprising a plurality of αTSHR ECD leucine rich regions (LRR); and

ϕ is a detectable label or absent.

In this study, L1-10 comprised

(i) a signal peptide (ψ)
(SEQ ID NO: 11)
MGWSLILLFLVAVATRVLS;
(ii) a binding molecule (β)
(SEQ ID NO: 12)
SAWSHPQFEKGGGSGGGSGGSSAWSHPQFEK;
(iii) a linker 1 (γ₁)
(SEQ ID NO: 13)
GGGS;
(iv) an expression enhancer (ε) of a maltose
binding protein having the sequence
(SEQ ID NO: 14)
KIEEGKLVIWINGDKGYNGLAEVGKKFEKDTGIKVTVEHPDKLEEKFPQV
AATGDGPDIIFWAHDRFGGYAQSGLLAEITPDKAFQDKLYPFTWDAVRYN
GKLIAYPIAVEALSLIYNKDLLPNPPKTWEEIPALDKELKAKGKSALMFN
LQEPYFTWPLIAADGGYAFKYENGKYDIKDVGVDNAGAKAGLTFLIDLIK
NKHMNADTDYSIAEAAFNKGETAMTINGPWAWSNIDTSKVNYGVTVLPTF
KGQPSKPFVGVLSAGINAASPNKELAKEFLENYLLTDEGLEAVNKDKPLG
AVALKSYEEELAKDPRIAATMENAQKGEIMPNIPQMSAFWYAVRTAVINA
ASGRQTVDEALKDAQT;
(v) a linker 2 (γ₂) comprises the polypeptide
(SEQ ID NO: 13)
GGGS;
(vi) a cleavable site (π) of the polypeptide
(SEQ ID NO: 15)
ENLYFQ;
(vii) a PEP sequence of the polypeptide
corresponding to aa20-275 of hTSHR:
(SEQ ID NO: 16)
GGMGCSSPPCECHQEEDFRVTCKDIQRIPSLPPSTQTLKLIETHLRTIPS
HAFSNLPNISRIYVSIDVTLQQLESHSFYNLSKVTHIEIRNTRNLTYIDP
DALKELPLLKFLGIFNTGLKMFPDLTKVYSTDIFFILEITDNPYMTSIPV
NAFQGLCNETLTLKLYNNGFTSVQGYAFNGTKLDAVYLNKNKYLTVIDKD
AFGGVYSGPSLLDVSQTSVTALPSKGLEHLKELIARNTWTLKKLPLSLSF
LHLTRA

The M22_NLuc_HisTag antibody protein amino acid sequence is as follows:

>mAB NLuc-His vH-IgG1
(SEQ ID NO: 17)
QVQLVQSGAEVKKPGESLKISCRGSGYRFTSYWINWVRQLPGKGLEWMGR
IDPTDSYTNYSPSFKGHVTVSADKSINTAYLQWSSLKASDTGMYYCARLE
PGYSSTWSVNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
KGGGSVFTLEDFVGDWRQTAGYNLDQVLEQGGVSSLFQNLGVSVTPIQRI
VLSGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERLIN
PDGSLLFRVTINGVTGWRLCERILAGGGSHHHHHHHHGS
>mAB NLuc-His VL-Lambda3
(SEQ ID NO: 18)
LTVLTQPPSVSGAPRQRVTISCSGNSSNIGNNAVNWYQQLPGKAPKLLIY
YDDQLPSGVSDRFSGSRSGTSASLAIRGLQSEDEADYYCTSWDDSLDSQL
FGGGTRLTVLGQPKAAPSVTLFPPSSEELQANKATLVCLISDFYPGAVTV
AWKADSSPVKAGVETTTPSKQSNNKYAASSYLSLTPEQWKSHKSYSCQVT
HEGSTVEKTVAPTECS

System performance was evaluated in an ELISA/microplate assay format, and luciferase enzyme activity of the fusion proteins was measured.

### Enzyme Luminescence Titration

Purified Nluc-fused M22 antibody or L1-10 antigen were sequentially diluted in PBS-BSA 0.1% solutions. 50 microliters of each dilution were distributed in a 96-well white microplate. 50 μL of furimazine diluted 200 times in PBS, 0.1% BSA were then added in each well. After a short incubation time (<3 min), luminescence was read on Perkin-Elmer VictorX 2030 Luminescence Reader (using PE Victor 2030 Workstation software).

ELISA assay procedure. Table 2 sets forth ELISA reagents used in assay development according to the present invention.

TABLE 2
ELISA reagents used in assay development

| Reagent | Source/Catalog # |
|---|---|
| M22, unlabeled | lot#1, Recombinantly expressed |
| | from HEK cells |
| L1-10, unlabeled | lot#11, Recombinantly expressed |
| | from HEK cells |
| M22_NLuc | mAb Nluc-His, 0.86 mg/mL, |
| | JS180124 |
| L1-10_NLuc | Strep-MBP-L1-10-Nluc, 0.20 mg/mL, |
| | JS180130 |
| 96-well microtiter | Thermo#7417-12, flat bottom, |
| plate, white | high binding |
| Coating buffer | PBS buffer, pH 7.2, #0110800 |
| Blocking Buffer: | StabilBlock (SurModics ST01-1000) |
| Wash buffer: | 1x PBS-0.05% Tween |
| Furimazie substrate | Promega #N113A, Nano-Glo assay |
| | substrate |
| Assay Buffer | Promega #N112A, Nano-Glo assay |
| | buffer |
| Streptavidin (SA) | Sekisui#STRE-70-4803 |
| Streptavidin magnetic beads | Thermo#88816 |
| Rabbit Anti-Mouse IgG | GE Healthcare#29-2152-81 |
| (RAM) | |
| StrepMAb, mouse | IBA# 2-1517-001 |
| monoclonal | |
| Anti-MBP, mouse | NEB#8032 |
| monoclonal | |

Plate Coating (using Microlite 2 White plates was performed as follows. Diluted plate coating protein was prepared in a PBS buffer. 50 μl or 100 μl/wells of 10 μg/mL antigen or antibody were added in coating buffer. The plate was sealed and incubated overnight at 4° C. No shaking was performed. The plates were then washed with PBST 4×, 250 μl/well, with a 5 second soak unless otherwise indicated. It was then blocked with 200 μl/well of blocking buffer, and the plate sealed and incubated for 1 hr at RT. The plate was then washed with PBST 4×.

Incubation steps were as follows. Appropriate diluted standards were prepared along with controls and samples in a Assay Diluent in a separate non-sticky plate. 50 μl or 100 μl of each antibody or antigen (at indicated concentrations) were pipetted to the plate wells. Plates were sealed and incubated for 60 minutes at RT (unless otherwise indicated). The plates were washed with PBST 4×. 50 μl/well of Furimazine substrate was pipetted and incubated for 3 minutes at RT. The plate was read on a Perkin-Elmer Victor X® 2030 Luminescence Reader (using PE Victor 2030 Workstation software).

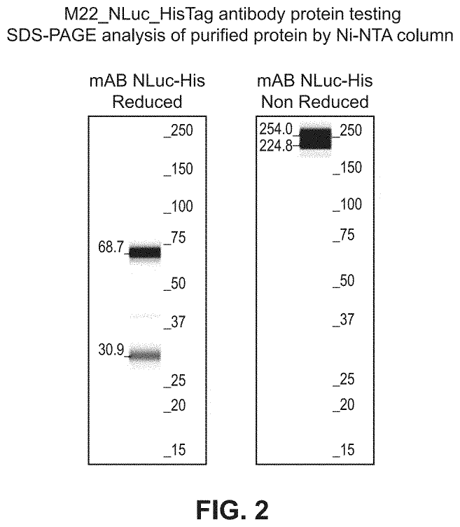

Protein characterization results demonstrated that the antibody fusion protein was purified as 70 kDa (heavy chain) and 30 kDa (Light chain) on reducing SDS-PAGE gel, and higher molecular bands were observed on a non-reduced gel (220 kDa bands). The purified protein showed as one major dominant peak on HPLC (Retention Time 5.3, corresponding to human IgG molecular weight). The results are set forth in FIG. 2.

### Example 4

M22_NLuc demonstrated broad linear range enzymatic Luminescence activity (up to 1.0E8 RLU). Good Signal/Noise ratio observed even at 1-10 pg/mL concentration. No decreased RLU signal was observed after substrate incubation for 5, 10 and 15 min.

L1-10 antigen was directly coated on microplate overnight. Serial diluted M22_NLuc was added to each well and incubated at room temperature for 30 min. The control wells were incubated with M22_Luc in the presence of 5 μg/mL M22 (unlabeled).

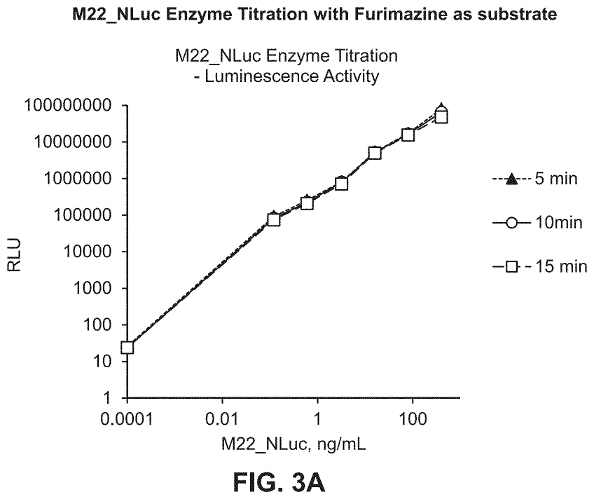

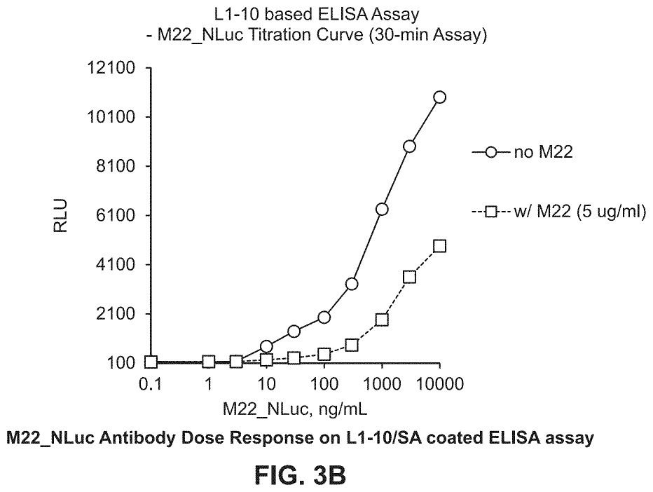

M22_NLuc demonstrated dose-response antigen binding activity in a 30-min assay (assay time was significantly shortened). Unlabeled M22 demonstrated specific inhibition to M22_Luc binding to coated L1-10. However, the sensitivity was very poor, consistent with previous studies that direct coating of L1-10 antigen would cause the inactivation of antigen-binding activity, thereby causing the loss of sensitivity. The results are set forth in FIGS. 3A and 3B.

### Example 5

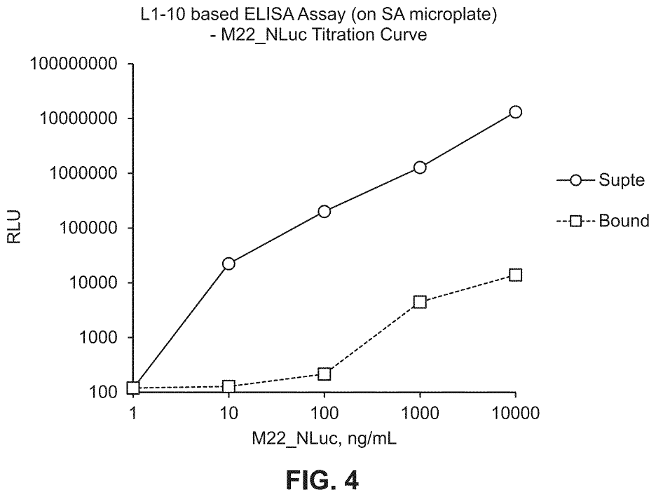

Streptavidin was coated as an additional step for L1-10 capture. Serial diluted M22_NLuc was added to each well and incubated at for 1 hr. Both the supernatants (Supte) and bound signal (Bound) were measured after substrate development. M22_NLuc demonstrated dose-response on SA coated plate. However, the sensitivity seemed very poor compared to the overall signal was added to each well as supernatants (Supte). This was due to the poor binding affinity between the SA and Strep tag on L1-10, thereby causing the loss of sensitivity. The results are shown in FIG. 4.

### Example 6

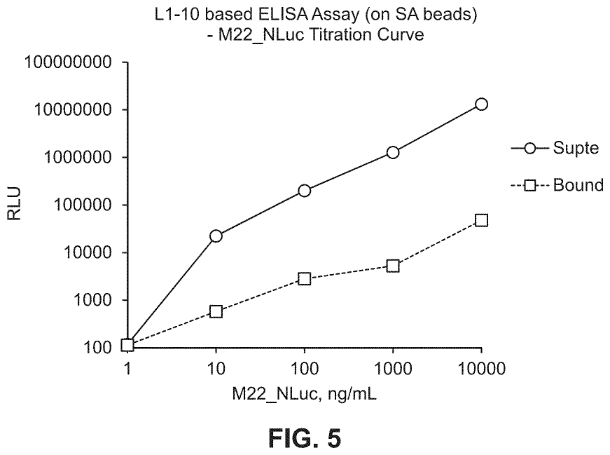

Streptavidin magnetic beads were used as an additional step for L1-10 capture. 1:10 diluted streptavidin magnetic beads were incubated with L1-10 and rotated for 1 hour. Washed by centrifugation (3000 rpm for 3 min) for 3 times. Serial diluted M22_NLuc was added to each tube and incubated at for 1 hr. Both the supernatants (Supte) and bound signal (Bound) were measured after substrate development. M22_NLuc demonstrated a dose-response on the SA beads assay. As above, the sensitivity would look poor compared to the overall signal as added to each well as supernatants (Supte). This was due to the poor binding affinity between the SA beads and Strep tag on L1-10, thereby causing the loss of sensitivity. These results are shown in FIG. 5.

### Example 7

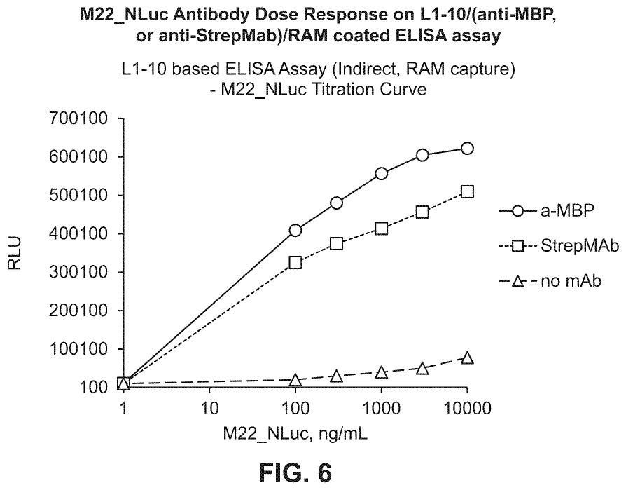

This study focused on the use of NanoLuc as a detection reagent and to develop an L1-10 assay. An indirect sandwich assay was tested using rabbit anti-Mouse (RAM, GE#29-2152-81) coated as an indirect capture for mouse monoclonals StrepMAb (IBA#2-1517-001), and Anti-MBP (NEB#E8032). The mouse monoclonal antibody served as the anchor for L1-10. Serial diluted M22_NLuc was added to each well and incubated at for 1 hr. M22_NLuc demonstrated good sensitivity over a broad range of RLU signals. Wide range of S/N was observed even at M22_NLuc concentration below <100 ng/mL. This demonstrated that RAM bound mouse monoclonal (anti-MBP or StrepMAb) well, capturing L1-10 antigen, and detected by M22-NLuc for needed sensitivity. The results are shown in FIG. 6.

### Example 8

### M22_NLuc Antibody Dose Response on L1-10/Anti-MBP Coated ELISA Assay

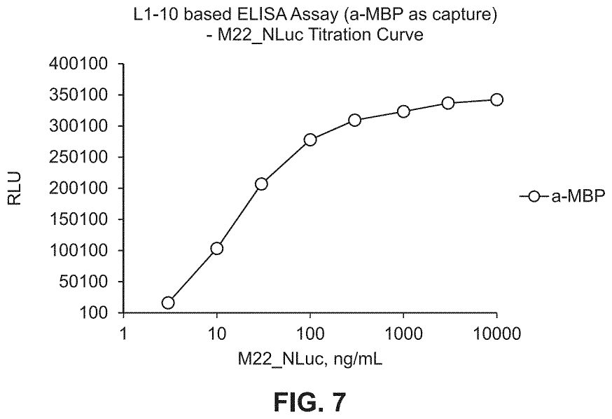

A standard sandwich assay was tested. Anti-MBP monoclonal antibody was coated as an direct capture for L1-10 antigen. Serial diluted M22_NLuc was added to each well and incubated for 1 hr. M22_NLuc demonstrated good sensitivity over a broad range of RLU signals. Wide range of S/N was observed even at M22_NLuc concentration well below 100 ng/mL. This demonstrated that mouse anti-MBP monoclonal bound L1-10 antigen well, detected by M22-NLuc for great sensitivity. The results are shown in FIG. 7.

### Example 9

Titration of M22 Competition (Dose Response) with M22_NLuc on L1-10/anti-MBP based ELISA assay. With fixed M22_NLuc concentration (starting at 120 ng/mL), unlabeled M22 was used for dose response titration. M22 demonstrated competition with M22_NLuc on this assay format. IC50 of M22 was determined as 3.8 IU/L & 2.1 IU/L (Note: 10 ng/mL=1 mIU/mL or 1 IU/L), respectively, using 120 or 40 ng/mL of M22_NLuc. With less and less M22_NLuc used in the assay system, the sensitivity would become better for detection. This was true for using 30 or 10 ng/mL of M22_NLuc in the next experiments. This is consistent with Data Analysis by Cheng-Prusoff Equation Ki=IC50/(1+([L]/Kd), where (L) is concentration of a ligand (M22-NLuc in this case). The Less [L] used in the system, the lower IC50 value would be. The better sensitivity can be reached using the minimal concentration of antibody or antigen.

[Ag] coated+[Ab-Luc] [symbol] Kd [Ag][Ab-Luc]

[Ag] coated +[Ab] [symbol] Ki [Ag][Ab]

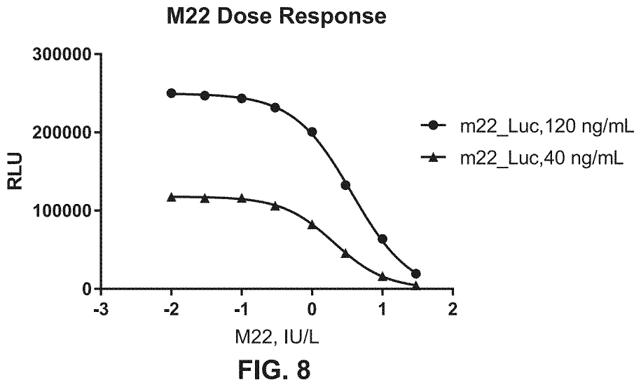

The results are shown in FIG. 8.

### Example 10

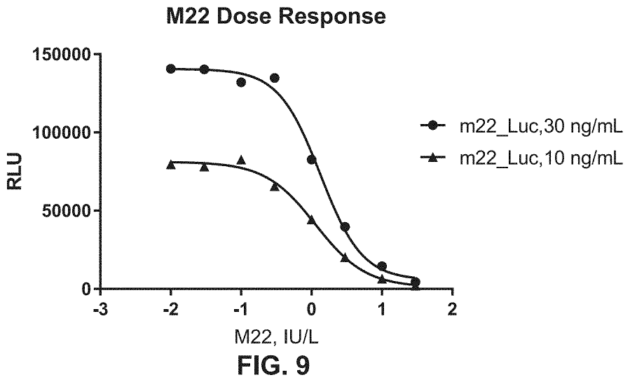

Titration of M22 Competition (Dose Response) with M22_NLuc on L1-10/anti-MBP based ELISA assay. With fixed M22_NLuc concentration (30 or 10 ng/mL), unlabeled M22 was used for dose response competition titration. IC50 of M22 (unlabeled) was determined as 1.5 IU/L & 1.0 IU/L, respectively, using 30 or 10 ng/mL of M22_NLuc. Therefore, the IC₅₀ of M22 decreased 3.8-fold (from 38 ng/mL to 10 ng/mL), when using less M22_NLuc (from 120 ng/mL to 10 ng/mL). Anti-MBP/L1-10 captured plate was stored at 4 C for 5 days, the same activity was observed. Results are set forth in FIG. 9.

### Example 11

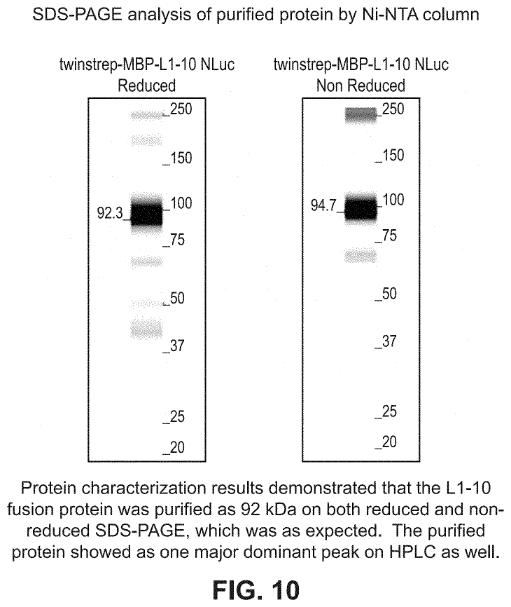

MBP_L1-10_NLuc Antigen protein testing was performed. FIG. 10 shows SDS-PAGE analysis of purified protein by Ni-NTA column.

### Example 12

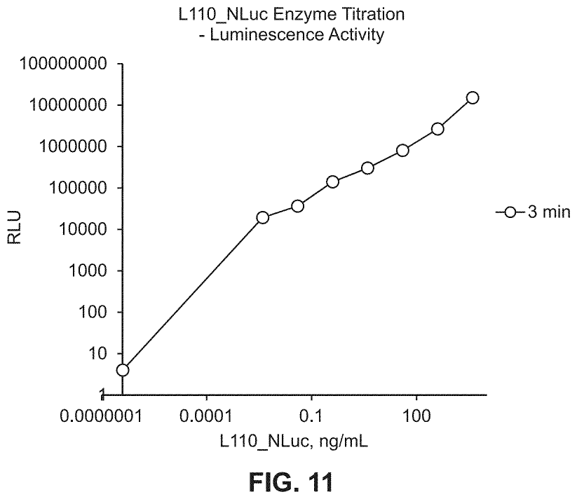

L1-10_NLuc Enzyme Titration with Furimazine substrate. L1-10_NLuc demonstrated broad linear range enzymatic Luminescence activity (up to 1.0E8 RLU). Good Signal/Noise ratio was observed even at 1-10 pg/mL concentration. Consistent RLU signal was observed after substrate incubation for 3 min. The results are set forth in FIG. 11.

### Example 13

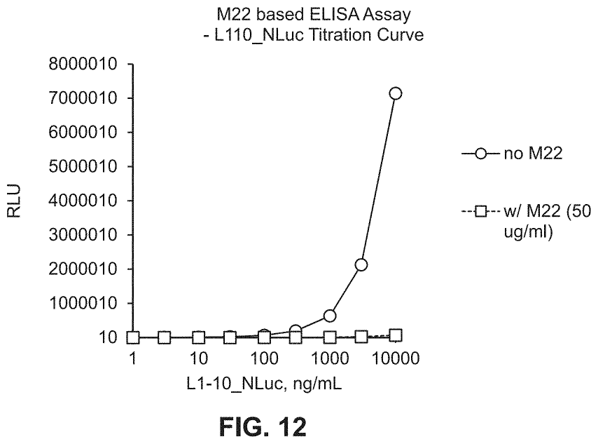

L1-10_NLuc Titration on M22 coated ELISA assay. M22 was directly coated on microplate overnight. Serial diluted L1-10_NLuc was added to each well and incubated at for 50 min. The control wells were incubated with L1-10_Luc in the presence of 50 μg/mL M22 (unlabeled). L1-10_NLuc demonstrated good sensitivity over a broad range of RLU signals (up to 7.0E6 RLU). Wide range of SN was observed even at L1-10_NLuc concentration well below 100 ng/mL. Therefore, the sensitivity would work out great when optimized to use less L1-10_NLuc. This demonstrated that one single step L1-10_NLuc assay was feasible and that specific dose-response binding activity was observed with exceptionally broad linear range. The results are set forth in FIG. 12.

### Example 14

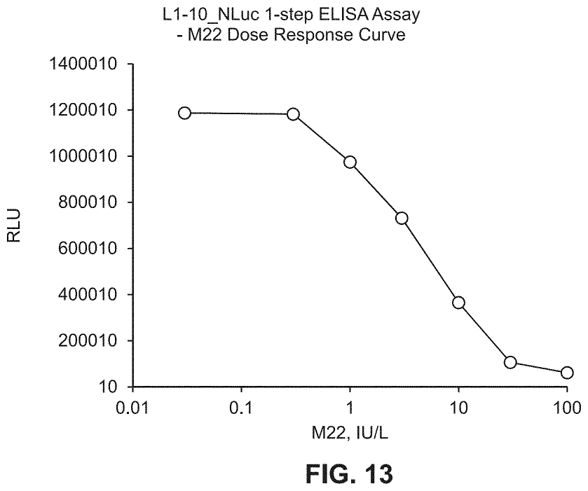

M22 Dose Response Curve on L1-10_NLuc/M22 based ELISA assay. M22 was coated on a microplate. Fixed concentration of L1-10_NLuc (with serial diluted M22) was added to each well and incubated at for 1 hr. With fixed L1-10_NLuc concentration (starting at 1 μg/mL), unlabeled M22 demonstrated good competition with L1-10_NLuc on this assay. M22 demonstrated good sensitivity over a broad range of RLU signals. IC50 of M22 was determined as 30 ng/mL (3 IU/L), using 1 μg/mL of L1-10_NLuc. The results are set forth in FIG. 13.

### Example 15

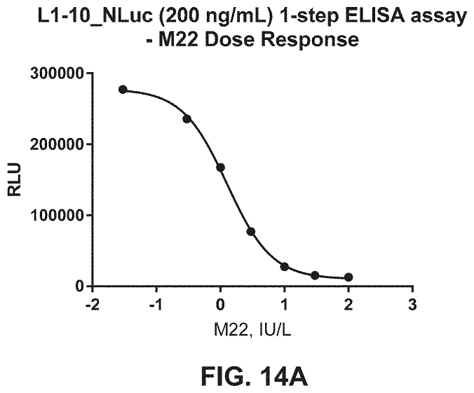

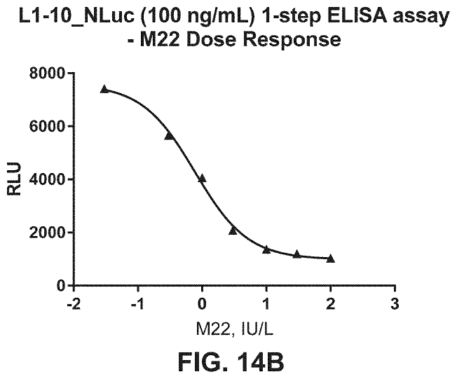

M22 Dose Response Curve on L1-10_NLuc/M22 based ELISA assay. M22 antibody was coated on microplate. Fixed concentration of L1-10_NLuc (with serial diluted M22) was added to each well and incubated at for 1 hr. With fixed L1-10_NLuc concentration (0.2 μg/mL or 0.1 μg/mL), unlabeled M22 demonstrated good competition with L1-10_NLuc on this assay. M22 demonstrated good sensitivity over a broad range of RLU signals. IC50 of M22 was determined as 12 ng/mL (1.2 IU/L) and 8 ng/mL (0.8 IU/L), using 0.2 μg/mL or 0.1 μg/mL of L1-10_NLuc, respectively. IC50 of M22 decreased 3.5-fold (from 30 ng/mL to 8 ng/mL), when using less L1-10_NLuc (from 1000 ng/mL to 100 ng/mL). The results are shown in FIG. 14A and 14B.

For M22_NLuc antibody protein, as the small protein NLuc was genetically fused with recombinant antibodies with stoichiometric ratio, this provided straightforward labeled reagents directly for assay development. The process resulted in maximal activity without antibody inactivation in comparison with the chemical labeling conjugation process. The broad luminescence linear dynamic range of NLuc activity allowed the L1-10 assay with enhanced sensitivity. At least 3-fold sensitivity was observed in comparison with the M22 dose-response curves measured by HRP conjugate approach herein. This sandwich format, with L1-10 captured by anti-MBP on plate, detected by M22_NLuc fusion, enabled one simple step quantitative ELISA in potentially less than 1-hour assay (as the L1-10 captured plates can be dried out and stably stored).

For L1-10_NLuc antigen proteins, as the small protein NLuc was genetically fused with recombinant antigen with stoichiometric ratio, this provided straightforward labeled reagents directly for assay development. The process resulted in maximal activity without antigen inactivation in comparison with the chemical labeling conjugation process. The direct immunoassay with engineered L1-10_NLuc enabled one single step quantitative Thyroid ELISA in less than 1-hour assay, with optimized sensitivity. The superior broad linear dynamic range allowed the L1-10 assay with enhanced sensitivity. At least 3-fold sensitivity was observed in comparison with the M22 dose-response curves determined with decreased concentrations of L1-10_NLuc. Potential applications of NanoLuc based technology platforms such as BRET can thus be used for further sensitive and quantitative POC assay development, in addition to the engineered L1-10_NLuc and M22_Nluc.

### Example 16

### Fluorescence Detection of TSHR on Cell Surface by M22 GFP Fab

CHO Cells were engineered with TSHR, 5×10⁶ cells/mL at a volume of 100 μL. The number of cells per condition was 5×10⁵. A control was designed using M22 Fabs at 0.95 to 30.4 μg/mL in a reaction buffer, and placed on ice for 1 hour. These were then incubated with mouse anti-human IgG1 Fc Antibody-Alexa Fluor 488 at a concentration of 42.5 μg/mL (i.e., 50% in excess to primary antibody) in reaction buffer and placed on ice for 1 hour. Testing with the M22 Fab-GFP (green fluorescence protein) was performed as follows.

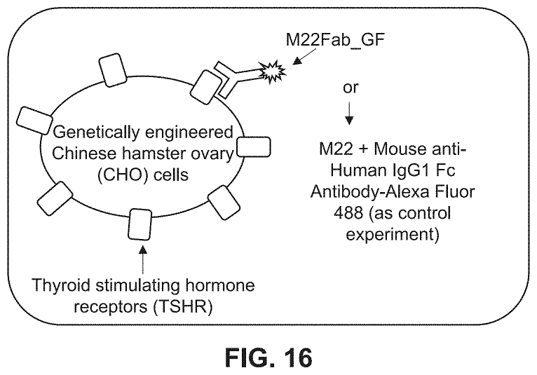

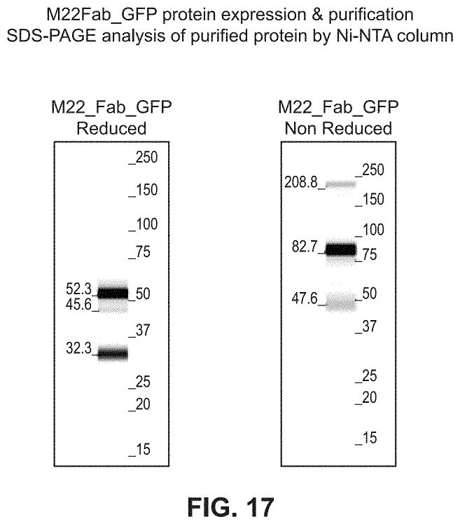

M22 Fab-GFP at a concentration of 0.9 to 45.5 μg/mL in reaction buffer were obtained and placed on ice for 1 hour. No secondary antibodies were required. The M22 Fab-GFP fusion was washed 3 times with centrifugation @300×g for 5 minutes each. Flow cytometry was performed on an Apogee Flow Systems cytometer and singlet cells gated for FITC/Alexa Fluor 488 fluorescence. Signals were recorded and dose response curves were plotted. A diagrammatic representation of the experiment is set forth in FIG. 16. The results of these experiments are set forth in FIGS. 17-19.

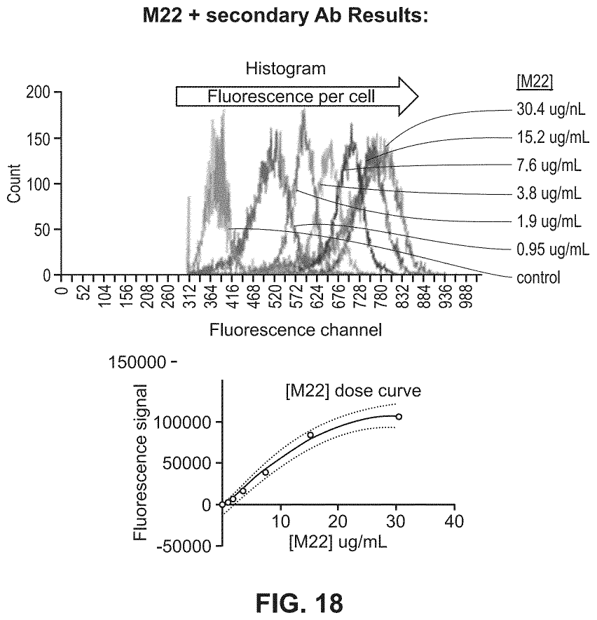

FIG. 17 sets forth M22Fab_GFP protein expression & purification via SDS-PAGE analysis of purified protein by Ni-NTA column. In FIG. 18, it can be seen that the negative control (primary Ab or secondary Ab alone) generated minimal signal as non-specific binding background. An increase with M22 with green fluorescence intensity was observed per cell.

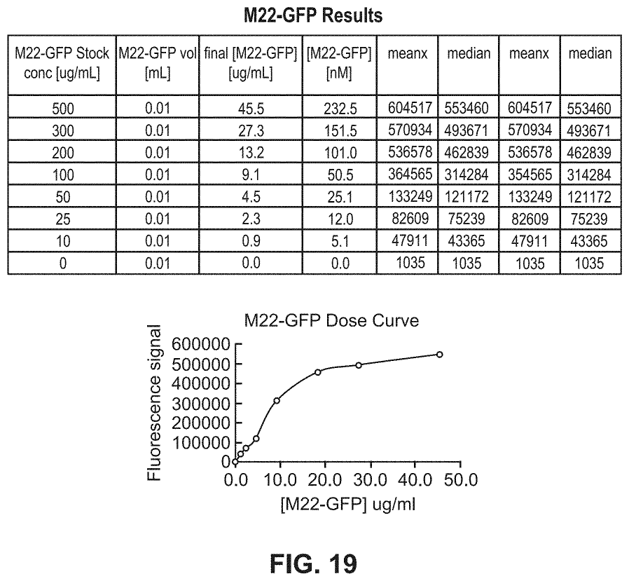

Fluorescence intensity peaked at around M22˜30 μg/mL with cells at 5×10 ⁶/mL. M22 showed an effective dose response when secondary antibody was used for detection of TSHR numbers on the cell surface. The negative control generated minimal signal as non-specific binding background. Increased M22-GFP resulted in measured green fluorescence intensity per cell. Fluorescence intensity plateaued at approximately M22-GFP 18 20 pg/mL with cells at 5×10⁶/mL. These results are set forth in FIG. 19. As can be seen, M22-GFP was equally effective as the combination of M22 with a secondary antibody for the detection of TSHR numbers on the cell surface. Secondary antibodies were therefore found to not be necessary. The results demonstrate a reduced variation in labeling and a reduction in the loss of cells and antibodies resulting in a more accurate measurement of receptors per cell.

### Example 17

### Lateral Flow Detection of Antibody Fusion

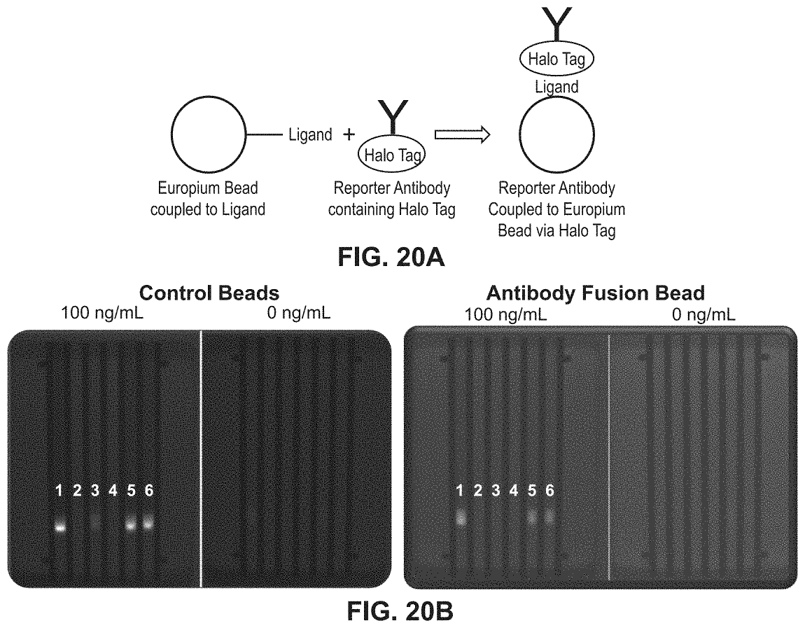

An antibody fusion of the present technology was analyzed for performance on a lateral flow immunoassay. The tested antibody fusion was an anti Flu A antibody fused to a Halo Tag (Promega) protein tag and conjugated to europium beads coupled to ligand (FIG. 20 A). The Halo Tag—ligand interaction is the basis for conjugation of the Halo Tag fused anti Flu A antibody with the ligand coupled europium bead. This antibody fusion conjugate was then spotted on nitrocellulose (channels 5 and 6); unmodified Flu A antibody was spotted on nitrocellulose as a positive control (channel 1); and ligand without antibody was spotted as a negative control (channels 2-4) of the nitrocellulose strips as shown in Table 3.

TABLE 3
Nitrocellulose Strip Channel Legend

| Channel | Capture |
|---|---|
| 1 | Control Bead: Flu A |
| 2 | Negative Control: Ligand 1 |
| 3 | Negative Control: Ligand 2 |
| 4 | Negative Control: Ligand 3 |
| 5 | Ligand-Flu A Ab (0.9 mg/mL)-1 |
| 6 | Ligand-Flu A Ab (0.6 mg/mL)-2 |

Each channel was spotted with a unique capture reagent (according to Table 3) with approximately 80-90nL of spotting solution. Spotted nitrocelluclose cards were then dried in a forced air oven for 5 minutes prior to dry storage.

Next, Flu A antigen was premixed with test beads coupled to Flu A control Ab (Control Beads) or FluA fusion Ab (Antibody Fusion Beads) prior to adding to the nitrocellulose strip. Antigen concentration was tested at two levels, 100 ng/mL and 0 ng/mL. 100 uL of premixed sample with beads was then added to the strip and allowed to run for 10 minutes prior to imaging

The results from the lateral flow immunoassays show that fluorescent signals were detected in channels 1, 5, and 6 for both control beads and antibody fusion beads (FIG. 20B). This result indicates the tested antibodies effectively bound to the capture antibody spotted in channels 1 (control Flu A antibody), 5 (0.9 mg/mL Flu A antibody fusion), and 6 (0.6 mg/mL Flu A antibody fusion). Fluorescent signals were absent from negative control ligand channels 2, 3, and 4. The absence of signal in these channels indicate specificity of the capture antibodies because the ligand spots in the absence of capture antibodies did not produce a positive signal. These results show that antibody fusion proteins comprising a Flu A/Halo Tag fusion perform properly in a lateral flow immunoassay. This further demonstrates utility of the presently described antibody fusions in lateral flow formats.

It is claimed:

1. A method of producing an antibody fusion, comprising:
a. obtaining a nucleic acid sequence of an antibody of interest, or fragment thereof;
b. operably linking said antibody of interest, or fragment thereof, nucleic acid to a nucleic acid sequence of a label of interest;
c. expressing the antibody or fragment thereof with the label of interest as an antibody fusion in a host cell; and
d. isolating said antibody fusion;
wherein the label of interest is selected from the group consisting of a luminescent label comprising a luciferase, a fluorescent label comprising at least one of GFP (green fluorescent proteins), RFP (red fluorescent proteins), CFP (cyan fluorescent proteins), or YFP (yellow fluorescent proteins), and a phosphatase label.

2. The method according to claim 1, wherein the antibody of interest is selected from the group consisting of anti-PCT antibodies, anti-thyroid TRAb, anti-lyme VlsE/C6, anti-OspC/10, anti-DbpA antibodies, and fragments thereof.

3. The method according to claim 1, wherein the luciferase is at least one of NLuc (NanoLuc), RLuc (RetinaLuc), and FLuc (FireflyLuc).

4. The method according to claim 1, wherein the phosphatase label comprises SEAP (Secreted Embryonic Alkaline Phosphatase).

5. The method according to claim 1, wherein the fluorescent label comprises GFP (green fluorescent proteins), RFP (red fluorescent protein), CFP (cyan fluorescent protein), or YFP (yellow fluorescent protein).

6. The method of claim 1, wherein the antibody of interest comprises an anti-PCT antibody or fragment thereof, and the label of interest comprises NanoLuc.

7. The method of claim 1, wherein the antibody of interest comprises an anti-PCT antibody or fragment thereof and the label of interest comprises SEAP.

8. The method of claim 1, wherein the antibody of interest comprises an anti-thyroid TRAb or fragment thereof and the label of interest comprises NLuc.

9. The method of claim 1, wherein the antibody of interest comprises a Lyme VlsE/C6 antibody, an OspC/10 antibody, or a DbpA antibody or fragments thereof.

10. The method of claim 1, wherein the antibody of interest comprises an M22 (TSHR-specific) antibody or fragment thereof, and the label of interest comprises a fluorescent protein comprising Green Fluorescent Protein (GFP).

11. The method of claim 1, wherein the antibody of interest comprises an M22 (TSHR-specific) antibody or fragment thereof, and the label of interest comprises a fluorescent protein comprising Red Fluorescent Protein (RFP).

12. The method of claim 1, wherein the antibody of interest comprises M22_NLuc, or fragment thereof, and wherein said antibody is paired with a second antibody comprising an RPE-anti human lgG, or fragment thereof.

13. An antibody fusion, comprising: an antibody or fragment thereof and a label of interest selected from the group consisting of:
a. a luciferase;
b. a fluorescent protein; and
c. SEAP (Secreted Embryonic Alkaline Phosphatase).

14. The antibody fusion according to claim 13, wherein the antibody is selected from the group consisting of anti-PCT antibodies, anti-thyroid TRAb, anti-lyme VlsE/C6, anti-OspC/10, anti-DbpA antibodies, and fragments thereof.

15. The antibody fusion according to claim 13, wherein the fluorescent protein is at least one of GFP (green fluorescent proteins), RFP (red fluorescent protein), CFP (cyan fluorescent protein), or YFP (yellow fluorescent protein).

16. The antibody fusion according to claim 13, wherein the luciferase is at least one of NLuc (NanoLuc), RLuc (RetinaLuc), and FLuc (FireflyLuc).

17. A method for diagnosing and/or detecting a disease or disorder of interest in a human subject, comprising:
a. providing an immunoassay comprising an antibody fusion according to claim 13;
b. contacting the immunoassay with a sample from a subject; and
c. detecting whether the antibody fusion binds a target in the sample to determine presence or absence of said disease or disorder.

18. The method of claim 17 wherein the label is SEAP.

19. The method of claim 17 wherein the label is a luminescent or fluorescent label.

20. The method of claim 19 wherein the luminescent or fluorescent label is selected from the group consisting of: a luciferase, GFP (green fluorescent proteins), RFP (red fluorescent protein), CFP (cyan fluorescent protein), or YFP (yellow fluorescent protein).

21. The method of claim 17 wherein the detecting step further comprises lateral flow detection.

22. The method of claim 17 further comprising a diagnostic testing system, wherein the diagnostic testing system comprises a lateral flow immunoassay with a fluorescently labelled antibody.

23. The method of claim 22 wherein the diagnostic testing system further comprises means to record and display instrument and user history data.

24. A plasmid comprising a nucleic acid sequence encoding the antibody fusion of claim 13.

25. A vector comprising the plasmid of claim 24.

26. A host cell comprising the vector of claim 25.

27. A kit comprising the antibody fusion of claim 13.
